(19) United States
(12) Patent Application Publication
(10) Pub. No.: US 2007/0004046 A1
(43) Pub. Date: Jan. 4, 2007

(54) Detection of analytes

(75) Inventor: Nicholas Abbott, Madison, WI (US)

Correspondence Address: J. Mitchell Jones;MEDLEN & CARROLL, LLP, Suite 350, 101 Howard Street, San Francisco, CA, 94105, US

(73) Assignee: Platypus Technologies, LLC, Madison, WI (US)

(21) Appl. No.: 11/173,122

(22) Filed: Jul. 1, 2005

(51) Int. Cl. G01N 33/00; C12Q 1/00; G02F 1/13

(52) U.S. Cl. 436/104; 435/4; 349/1

(57) ABSTRACT

The present invention relates to the field of detection of analytes, and in particular to detection of organophosphates using a liquid crystal assay format and a variety of analytes in stand-off detection formats utilizing liquid crystals as part of reporting system. The present devices find use in detecting cumulative exposure to organophosphates in the aerosol phase.

This application claims the benefit of U.S. Provisional Application 60/525,275, filed Jul. 2, 2004.

This invention was made in part with government support under SBIR Grant No. 5 R43 ES11217-02 awarded by NIH-NIEHS. The Government has certain rights in the invention.

### FIELD OF THE INVENTION

The present invention relates to the field of detection of analytes, and in particular to detection of organophosphates using a liquid crystal assay format and a variety of analytes in stand-off detection formats utilizing liquid crystals as part of reporting system.

### BACKGROUND OF THE INVENTION

Organophosphates (OPs) are used extensively throughout the world as pesticides. In 1998-1999, 91 million pounds of organophosphate insecticides were used in the United States, accounting for 72% of all insecticide used that year. The most commonly used OP insecticides' active ingredients, ranked in usage from 1-5, were: Malathion, Chlorpyrifos, Tebufos, Diazinon and methyl Parathion (EPA, 1998-1999). There are 37 organophosphate pesticides registered with the EPA. Approximately one quarter of the usage in the United States is for non-agricultural applications; home and garden, commercial buildings and mosquito control.

OPs are present in the environment as residues on foods, contaminants in soil, solutes in groundwater and drinking water and volatilized in the air. OPs are efficiently absorbed by inhalation, skin penetration and by ingestion (Cherimisinoff and King, 1999, Hallenbeck and Cunningham, 1985). Everyone, rural, suburban and urban dweller alike, has exposure to these compounds. The National Health and Nutrition Examination Survey (NHANES III, 1988-1994) detected metabolites of two OP pesticides in the urine of 82% of the 900 tested adult volunteers from all regions of the country.

The number of registered uses of these OPs have been significantly reduced in recent years, particularly those OPs used for residential treatments, due to concerns about health risks. Malathion and Diazinon, however, are still widely used in agriculture, and have many outdoor residential applications. Malathion, for example, is licensed for commercial agricultural use on more than 100 food and feed crops (vegetables, fruits, grains, and fodder); for use around the home on vegetables, turf, fruit and ornamental trees, and to kill pests attacking outdoor dwellings; for use by regional pest control programs (boll weevils, med fly, and mosquito control), and as a treatment for head lice and their ova (USEPA 2003). EPA considers Parathion a “restricted use pesticide,” that has few uses, all agricultural.

There is increasing concern about the potential effects of pesticides on children's health. Much of the concern is driven by mounting evidence from both animal toxicological studies and epidemiological investigations that children and adults may suffer adverse health effects from chronic low level exposure to organophosphate (OP) and other pesticides. Furthermore, it is now widely recognized that health risk assessments should take special account of children because they may be both more exposed and more biologically susceptible than adults (Guzelian et al. 1992; NRC 1993). Among the reasons children may be at potentially greater risk are their lower body weights, developing organs, higher metabolic rates, and unique behavior patterns.

The Food Quality Protection Act (FQPA) of 1996 (P.L. 104-170) requires that children's exposure to pesticides be evaluated for all potential pathways, both dietary (i.e., consumption of food and beverages) and non-dietary (i.e., intake of pesticides in air, water, and soil or dust). The FQPA codified the need for more and better exposure data to help in the process of risk-based decision making, and mandated an examination of aggregate (all potential routes of exposure) and cumulative (all compounds with a common mechanism) exposures. This means that environmental risk assessors must quantify pesticide exposure by multiple routes: i.e., inhalation; dermal absorption; dietary and non-dietary (soil and house dust) ingestion.

One of the major problems in completing these assessments is the lack of monitoring data to estimate exposure from non-dietary routes.

### SUMMARY OF THE INVENTION

The present invention relates to the field of detection of analytes, and in particular to detection of organophosphates using a liquid crystal assay format and a variety of analytes in stand-off detection formats utilizing liquid crystals as part of reporting system. Accordingly, in some embodiments, the present invention provides methods of remotely detecting an analyte comprising: a) providing a plurality of liquid crystal assay devices comprising a first surface displaying a recognition moiety, the first surface in contact with a liquid crystal; b) exposing the plurality of liquid crystal assay devices to a sample suspected of containing the analyte; and c) simultaneously irradiating the plurality of liquid crystal assay devices under conditions such that radiation returned from the plurality of liquid crystal assay devices is indicative of a change in orientation of the liquid crystal in the assay devices caused by interaction of the analyte with the recognition moieties. In some embodiments, the irradiating step is performed by exposure to electromagnetic radiation. The present invention is not limited to the use of any particular type of electromagnetic radiation. Indeed, the use of a variety of types of electromagnetic radiation is contemplated, including, but not limited to visible light, x-ray radiation, UV radiation, infrared radiation, and radio frequency radiation. In some embodiments, the radiation returned from the devices is measured by a detector. The present invention is not limited to any particular type of detection. Indeed, a variety of types of detection are useful, including, but not limited to, infrared spectroscopy, raman spectroscopy, x-ray spectroscopy, visible light spectroscopy, ultraviolet spectroscopy, spectroscopy of radio frequency radiation, and combinations thereof. In some embodiments, the radiation returned from the device exhibits a peak wavelength that is different in the presence of an analyte than in the absence of an analyte. In other embodiments, the radiation returned from the device exhibits a spectrum that is different in the presence of an analyte than in the absence of an analyte. In still other embodiments, the radiation returned from the device exhibits a change in the intensity of the peak of the spectrum emitted from the device.

In some embodiments, the liquid crystal assay device comprises a fluorophore. In further embodiments, the irradiating step excites the fluorophore and wherein the wavelength of light emitted by the fluorophore is different in the presence an analyte than in the absence of an analyte. In some embodiments, the surface comprises a semiconductor quantum dot that fluoresces when exposed to radiation. In further embodiments, when the surface is irradiated with ultraviolet radiation and the wavelength of light emitted by the surface is different in the presence an analyte than in the absence of an analyte. In some embodiments, the surface comprises periodic lines displaying the recognition moiety. In further embodiments, the binding of the analyte to the recognition moiety causes a change in the light returned from the device upon irradiation.

The methods of the present invention are not limited to the use of any particular recognition moiety. Indeed, the use of a variety of different recognition moieties is contemplated, including, but not limited to, metal ions, metal-binding ligands, nucleic acids, polypeptides, proteins, acids, bases, antibodies, enzymes, and combinations thereof. The methods of the present invention are not limited to the detection of any particular analyte. Indeed, the detection of a variety of analytes is contemplated, including, but not limited to organophosphates, explosive agents, chemical warfare agents, polypeptides, polynucleotides, toxins, volatile organic compounds, viruses, and microorganisms. In some preferred embodiments, the organophosphates are selected from the group consisting of pesticides and chemical warfare agents.

The present invention is not limited to the use of devices with particular types of surfaces. Indeed, the use of a variety of surface materials is contemplated, including, but not limited to gold and silicon. The present invention is not limited to any particular liquid crystal assay device format. Indeed, a variety of formats are contemplated, including, but not limited to planar, spherical, and cylindrical formats. In some embodiments, the liquid crystal assay device comprises porous silicon, and wherein the recognition moieties and the liquid crystal are contained within pores in the porous silicon. The present invention is not limited to the use any particular type of mesogen. Indeed, the use of a variety of mesogens is contemplated, including, but not limited to E7, MLC, 5CB (4-n-pentyl-4′-cyanobiphenyl), and 8CB (4-cyano-4′octylbiphenyl). The present invention is not limited to assay devices of any particular size. Indeed, a variety of sizes are contemplated. In some embodiments, the liquid crystal assay devices are less than 1 cm in width and length, respectively. In other embodiments, the liquid crystal assay devices are less than 1 mm in width and length, respectively.

In some preferred embodiments, the liquid crystal assay devices are irradiated by a remote radiation source. The present invention is not limited to remote detection from any particular distance. In some embodiments, the remote radiation source is greater than 10 meters from the liquid crystal assay device. In other embodiments, the remote radiation source is greater than 100 meters from the liquid crystal assay device. In still further embodiments, the remote radiation source is greater than 1000 meters from the liquid crystal assay device. In some embodiments, the plurality of liquid crystal assay devices are deployed and irradiated in the atmosphere. The present invention is not limited to any particular method of deployment. Indeed, a variety of methods of deployment are contemplated, including, but not limited to, planes, rockets, balloons, and helicopters. The present invention is not limited to the analysis of any particular type of sample. Indeed, analysis of a variety of samples is contemplated, including but not limited to, samples selected from the group consisting of atmosphere, gas, vapor, mist, and liquid.

In some embodiments, the liquid crystal assay devices comprise a second surface opposed to the first surface. In further embodiments, the first and second surfaces are reflective. In still further embodiments, the second surface displays a recognition moiety. In still other embodiments, the first surface and the second surface form a Fabry-Perot filter.

In other embodiments, the present invention provides assay devices comprising an interior cylindrical surface and an exterior cylindrical surface, the interior and exterior cylindrical surfaces opposed to one another to form a chamber there between, wherein the at least one of the interior and exterior cylindrical surfaces displays a recognition moiety and wherein the chamber is substantially filled with a liquid crystal. In some embodiments, the interior surface and exterior cylindrical surfaces are reflective. In some preferred embodiments, the interior surface comprises gold. In further preferred embodiments, the exterior surface comprises nanoporous gold. The present invention is not limited to the use any particular type of mesogen. Indeed, the use of a variety of mesogens is contemplated, including, but not limited to E7, MLC, 5CB (4-n-pentyl-4′-cyanobiphenyl), and 8CB (4-cyano-4′octylbiphenyl). The devices of the present invention are not limited to the use of any particular recognition moiety. Indeed, the use of a variety of different recognition moieties is contemplated, including, but not limited to, metal ions, metal-binding ligands, nucleic acids, polypeptides, proteins, acids, bases, antibodies, enzymes, and combinations thereof. The present invention is not limited to cylinders of any particular dimension. In some preferred embodiments, the length of the assay device as measured along the axis of the cylinder is less than about 1 cm. In further preferred embodiments, the width of the assay device as measured perpendicular to the axis of the cylinder is less than about 2000 microns. In still further preferred embodiments, the interior and exterior cylindrical surfaces form a Fabry-Perot filter.

In still further embodiments, the present invention provides assay devices comprising an interior spherical surface and an exterior spherical surface, the interior and exterior spherical surfaces opposed to one another to form a chamber there between, wherein the at least one of the interior and exterior cylindrical surfaces displays a recognition moiety and wherein the chamber is substantially filled with a liquid crystal.

In some preferred embodiments, the interior surface comprises gold. In further preferred embodiments, the exterior surface comprises nanoporous gold. The present invention is not limited to the use any particular type of mesogen. Indeed, the use of a variety of mesogens is contemplated, including, but not limited to E7, MLC, 5CB (4-n-pentyl-4′-cyanobiphenyl), and 8CB (4-cyano-4′octylbiphenyl). The devices of the present invention are not limited to the use of any particular recognition moiety. Indeed, the use of a variety of different recognition moieties is contemplated, including, but not limited to, metal ions, metal-binding ligands, nucleic acids, polypeptides, proteins, acids, bases, antibodies, enzymes, and combinations thereof. The present invention is not limited to cylinders of any particular dimension. In some preferred embodiments, the diameter of the assay device is less than about 1 cm. In still further preferred embodiments, the interior and exterior cylindrical surfaces form a Fabry-Perot filter.

In still other embodiments, the present invention provides assay devices comprising a porous silicon having pores therein, wherein the pores have a pore surface displaying a recognition moiety and wherein the pores are substantially filled with a liquid crystal. In some preferred embodiments, the pore surfaces are reflective. The present invention is not limited to the use any particular type of mesogen. Indeed, the use of a variety of mesogens is contemplated, including, but not limited to E7, MLC, 5CB (4-n-pentyl-4′-cyanobiphenyl), and 8CB (4-cyano-4′octylbiphenyl). The devices of the present invention are not limited to the use of any particular recognition moiety. Indeed, the use of a variety of different recognition moieties is contemplated, including, but not limited to, metal ions, metal-binding ligands, nucleic acids, polypeptides, proteins, acids, bases, antibodies, enzymes, and combinations thereof. The present invention is not limited to devices of any particular dimensions. In some embodiments, the surface area of the assay device is less than about 1 cm. In some preferred embodiments, the pores form a rugate filter.

In other embodiments, the present invention provides systems for remotely detecting an analyte comprising: a) a plurality of liquid crystal assay devices comprising a first surface displaying a recognition moiety, the first surface in contact with a liquid crystal; b) a radiation source remote from the plurality of liquid crystal assay devices; and c) a detector configured to receive a signal from the plurality of assay devices upon radiation of the plurality of assay devices by the radiation source.

In some embodiments, the radiation source emits electromagnetic radiation. The present invention is not limited to the use of any particular type of electromagnetic radiation. Indeed, the use of a variety of types of electromagnetic radiation is contemplated, including, but not limited to visible light, x-ray radiation, UV radiation, infrared radiation, and radio frequency radiation. The present invention is not limited to any particular type of detector. Indeed, a variety of types of detectors are useful, including, but not limited to, infrared spectroscopes, raman spectroscopes, x-ray spectroscopes, visible light spectroscopes, ultraviolet spectroscopes, radio frequency radiation spectroscopes, and combinations thereof. In some embodiments, the radiation returned from the device exhibits a peak wavelength that is different in the presence of an analyte than in the absence of an analyte. In other embodiments, the radiation returned from the device exhibits a spectrum that is different in the presence of an analyte than in the absence of an analyte. In still other embodiments, the radiation returned from the device exhibits a change in the intensity of the peak of the spectrum emitted from the device.

In some embodiments, the liquid crystal assay device comprises a fluorophore. In further embodiments, the irradiating step excites the fluorophore and wherein the wavelength of light emitted by the fluorophore is different in the presence an analyte than in the absence of an analyte. In some embodiments, the surface comprises a semiconductor quantum dot that fluoresces when exposed to radiation. In further embodiments, when the surface is irradiated with ultraviolet radiation and the wavelength of light emitted by the surface is different in the presence an analyte than in the absence of an analyte. In some embodiments, the surface comprises periodic lines displaying the recognition moiety. In further embodiments, the binding of the analyte to the recognition moiety causes a change in the light returned from the device upon irradiation.

The systems of the present invention are not limited to the use of any particular recognition moiety. Indeed, the use of a variety of different recognition moieties is contemplated, including, but not limited to, metal ions, metal-binding ligands, nucleic acids, polypeptides, proteins, acids, bases, antibodies, enzymes, and combinations thereof. The systems of the present invention are not limited to the detection of any particular analyte. Indeed, the detection of a variety of analytes is contemplated, including, but not limited to organophosphates, explosive agents, chemical warfare agents, polypeptides, polynucleotides, toxins, volatile organic compounds, viruses, and microorganisms. In some preferred embodiments, the organophosphates are selected from the group consisting of pesticides and chemical warfare agents.

The present invention is not limited to the use of devices with particular types of surfaces. Indeed, the use of a variety of surface materials is contemplated, including, but not limited to gold and silicon. The present invention is not limited to any particular liquid crystal assay device format. Indeed, a variety of formats are contemplated, including, but not limited to planar, spherical, and cylindrical formats. In some embodiments, the liquid crystal assay device comprises porous silicon, and wherein the recognition moieties and the liquid crystal are contained within pores in the porous silicon. The present invention is not limited to the use any particular type of mesogen. Indeed, the use of a variety of mesogens is contemplated, including, but not limited to E7, MLC, 5CB (4-n-pentyl-4′-cyanobiphenyl), and 8CB (4-cyano-4′octylbiphenyl). The present invention is not limited to assay devices of any particular size. Indeed, a variety of sizes are contemplated. In some embodiments, the liquid crystal assay devices are less than 1 cm in width and length, respectively. In other embodiments, the liquid crystal assay devices are less than 1 mm in width and length, respectively.

In some preferred embodiments, the radiation source is remote from the liquid crystal assay devices. The present invention is not limited to remote detection from any particular distance. In some embodiments, the remote radiation source is greater than 10 meters from the liquid crystal assay device. In other embodiments, the remote radiation source is greater than 100 meters from the liquid crystal assay device. In still further embodiments, the remote radiation source is greater than 1000 meters from the liquid crystal assay device. In some embodiments, the plurality of liquid crystal assay devices are deployed and irradiated in the atmosphere. The systems of the present invention are not limited to any particular method of deployment. Indeed, a variety of methods of deployment systems are contemplated, including, but not limited to, planes, rockets, balloons, and helicopters. The present invention is not limited to the analysis of any particular type of sample. Indeed, analysis of a variety of samples is contemplated, including but not limited to, samples selected from the group consisting of atmosphere, gas, vapor, mist, and liquid.

In some embodiments, the liquid crystal assay devices comprise a second surface opposed to the first surface. In further embodiments, the first and second surfaces are reflective. In still further embodiments, the second surface displays a recognition moiety. In still other embodiments, the first surface and the second surface form a Fabry-Perot filter.

In some embodiments, the present invention provides methods of assaying cumulative exposure to organophosphates comprising: a) providing a device comprising a liquid crystal, the liquid crystal between a first surface and a second surface, the first surface comprising an organic layer in contact with the first surface, the organic layer having immobilized thereon at least one metal ion, the device having an opening therein; and b) exposing the device to a sample suspected of containing organophosphates, wherein cumulative exposure to organophosphates is indicated by a change in the orientation of the liquid crystal identified as wavefront advancing from the opening. The present invention is not limited to the use of any particular metal ion. Indeed, the use of a variety of metal ions is contemplated, including, but not limited to those selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺. In some embodiments, the metal ions are arranged in an array on the device surface. In some preferred embodiments, the identity of a particular organophosphate is discernable from the pattern of liquid crystal orientation on the array.

In some embodiments, the sample suspected of containing organophosphates contains organophosphates in aerosol phase. In still other embodiments, the exposing step is from about 1 hour to about 30 days in length. In other embodiments, the present invention provides methods of identifying a particular organophosphate comprising: a) providing a substrate comprising at least two detection regions having at least two different metal ions immobilized thereon; and b) exposing the device to a sample suspected of containing an organophosphate; and c) determining the identity of the organophosphate by examining the change of liquid crystal orientation in said detection regions. In some embodiments, the liquid crystal overlaying the detection regions is disordered in the presence of organophosphates. In further embodiments, the detection region has immobilized thereon a plurality of different metal ions selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺. In some embodiments, the sample suspected of containing organophosphates contains organophosphates in aerosol phase. In further embodiments, the exposing step is from about 1 hour to about 30 days in length. In still further embodiments, the change in orientation is indicated by an advancing wavefront. In still other embodiments, the advancement of the wavefront correlates to exposure to organophosphates. In some embodiments, the detection region comprises an organic layer and said metal ion is immobilized via said organic layer. In further embodiments, the organic layer comprises a member selected from the group consisting of 11-mercaptoundecanoic acid, 4-aminothiophenol, and mercaptobenzoic acid. In other embodiments, the methods comprise further providing a second substrate opposed to said first substrate, wherein said first and second substrates form a chamber for receiving a liquid crystal.

In some embodiments of the present invention, a device is provided that comprises at least a first substrate having a surface, the substrate comprising at least first and second detection regions on the surface, wherein the first and second detection regions comprise an organic layer and a metal ion immobilized on the organic layer and wherein said metal ions on the first and second detection regions are different. In some embodiments, the organic layer comprises a member selected from the group consisting of 11-mercaptoundecanoic acid, 4-aminothiophenol, and mercaptobenzoic acid. In further embodiments, the first detection region comprises a metal ion selected from the group consisting of Al³⁺, Ag¹⁺, Ba³⁺, Cd²⁺, Ce³⁺, Co²⁺, Cr⁺, Eu³⁺, Fe²⁺, Fe³⁺, Ga³⁺, In³⁺, Mn²⁺, Ni²⁺, Pb²⁺, Pr³⁺, and Zn²⁺ and the second detection region comprises a different metal ion selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺. In still other embodiments, the detection region is configured to contain a liquid crystal. In some preferred embodiments, the detection region is in a well. In some embodiments, the devices further comprise a second substrate opposed to said first substrate, wherein said first and second substrates form a chamber for receiving a liquid crystal.

In still further embodiments, the present invention provides devices comprising: at least a first substrate having a surface, said substrate further comprising at least a first detection region on said surface, wherein said detection region comprises a recognition moiety; a liquid crystal in contact with the first substrate; and a housing having an opening therein, said substrate configured in said housing so that said detection region is exposed to the atmosphere through said opening. In some embodiments, the housing is movable between an exposure position wherein the detection region is exposed to the atmosphere through the opening and a reading position wherein said detection region is substantially closed off to the atmosphere. In some embodiments, the devices further comprise a filter in the opening. In some preferred embodiments, the filter is an aerosol filter. In some embodiments, the recognition moiety is a metal ion. In further embodiments, the metal ion is selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺. In some preferred embodiments, the metal ion is immobilized on the detection region via an organic layer. In some embodiments, the organic layer comprises a member selected from the group consisting of 11-mercaptoundecanoic acid, 4-aminothiophenol, and mercaptobenzoic acid. In some preferred embodiments, the detection region is configured to contain a liquid crystal. In some embodiments, the detection region is in a well. In still other embodiments, the devices further comprise a second substrate opposed to the first substrate, wherein the first and second substrates form a chamber for receiving a liquid crystal. In some embodiments, the first substrate comprises a plurality of distinct detection regions. In further embodiments, the plurality of distinct detection regions comprises at least two different recognition moieties.

### DESCRIPTION OF THE FIGURES

FIG. 1 provides a depiction and pictures of an assay device before and after response to an organophosphate.

FIG. 2 provides the structures of DMMP, malathion, and diazinon.

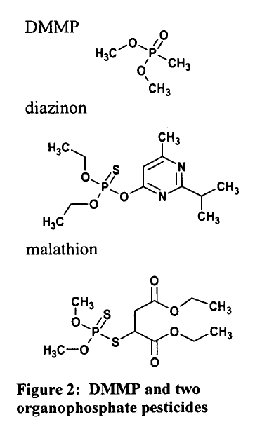

FIG. 3 provides a depiction of categorization of the different magnitudes of response liquid crystal assay devices. Images of optical cells were captured with a digital camera and analyzed with Scion software. The results were expressed as the number of pixels that corresponded to the length of the planar front. Strong (S, 41-93 pixels), moderate (M, 21-40 pixels), weak (W, 5-20 pixels), very weak (VW, less than 5 pixels) or none.

FIG. 4 provides examples of multi-metal array fingerprints for different compounds. Each compound causes a different set of responses from this group of metals. The response to most compounds is easy to differentiate from one another.

FIG. 5 provides an illustration of an open cell before and after response to analyte. In the open cell, the analyte diffuses through the top of the cell. After exposure to the analyte, the liquid crystal quickly changes from homeotropic to planar throughout the cell.

FIG. 6 provides data demonstrating selectivity of response to water and DMMP. Optical images of a film of E7 supported on a SAM of MUA that was pretreated by immersion into an ethanolic solution of 2 mM Eu(ClO₄)₃ (A) after fabrication; (B) after 30 min exposure to 85% humidity; (C) after subsequent exposure to 4 ppm DMMP for 10 sec.

FIG. 7 provides data demonstrating an increased stability to high humidity with E7/MLC mixture. LC films are tolerant to exposure to 85% humidity for 20 hrs and retain responsiveness to DMMP. Optical images of a film of a 1:1 (v/v) mixture of E7 and MLC 15000-000 supported on a an MUA SAM that was pretreated by immersion into an ethanol solution of 2 mM In(ClO₄)₃ A) after fabrication; B) after exposure to 85% humidity for 1 h; C) after exposure to 85% humidity for 20 h; D) after subsequent exposure to 4 ppm DMMP for 45 sec.

FIG. 8 provides a graphical depiction of the dynamics of DMMP response tailored by metal selection. Open cell response to 4 ppm DMMP. In³⁺ and Eu³⁺ respond rapidly, whereas Mn²⁺ shows no response under the same conditions, demonstrating the selective nature of the 3 metals used in an open cell geometry.

FIG. 9 provides data demonstrating the cumulative exposure of a 1 mM In³⁺ cell to 4 ppm DMMP.

FIG. 10 provides a graphical depiction of the cumulative exposures of In³⁺ cells to DMMP at different concentrations.

FIG. 11 provides an illustration of a multi-metal array cell.

FIG. 12 provides a graphical depiction of the response of a multi-metal array exposed to 4 ppm DMMP for 4 hours.

FIG. 13 provides data demonstrating the reversibility of binding. A gold coated slide was treated with MUA for 1 hour. A 1 mM ethanolic In(ClO₄)₃ solution was spin coated on the surface and a film of E7 was applied to form the open cell. The reversibility of the E7 alignment was tested by exposing the cell to 80 ppb DMMP for 3 min and then purging with N₂. (A) graph of the DMMP response; (B) before exposure; (C) at 3 min; (D) at 4.5 min.

FIG. 14 provides a graphical depiction of response rate Δ(ΔS/S)/Δt at different DMMP concentrations. A film of E7 supported on a SAM formed from MUA that was pretreated by immersion into an ethanol solution of 2 mM In(ClO₄)₃ and sequentially exposed to different DMMP concentrations.

FIG. 15 provides data demonstrating the response of a device to long-term exposure to Maxide® Diazinon. Cumulative exposure to ˜5 ppb commercial-brand Maxide® Diazinon. Images: (A) before exposure (B) 8 day exposure (C) 11 day exposure (D) 15 day exposure and (E) 22 day exposure.

FIG. 16 provides data demonstrating the effect of metal ions and concentration on response rate by an assay device.

FIG. 17 provides control cells that confirm positive response to diazinon. Optical cells were exposed to (A) ambient atmosphere for 10 days and did not show response and (B) pure diazinon for six days showing positive results. All control cells were exposed at ˜50% humidity.

FIG. 18 provides data demonstrating the response of a device to long-term exposure to Ortho® Malathion. Cumulative exposure to ˜0.5 ppb commercial-brand Ortho® Malathion. Pictures (A) through (D) represent 1 mM Pb with 1 mM MBA (A) before exposure (B) 7 day exposure (C) 12 day exposure and (D) 19 day exposure.

FIG. 19 provides a graphical depiction of the influence of the ligand used to immobilize the metal ions on the response of the liquid crystal to DMMP. Varying the ligand in the self-assembled monolayer has a large effect on the response of the optical cells to DMMP.

FIG. 20 provides a view of a template for cutting glass slides. The glass slides slip beneath the template.

FIG. 21 provides an image of a device converted to binary for analysis using % white command.

FIG. 22 provides a graph and picture used for plot profile analysis.

### DEFINITIONS

As used herein, the term “organophosphate” refers to phosphorous containing organic compounds.

As used herein, the term “wavefront” refers to a line of demarcation that is observable between a region of ordered liquid crystal and a region of disordered liquid crystal. In many cases, the wavefront is visually detectable. However, the location of the wavefront can also be detected by image analysis procedures.

As used herein, the term “ligand” refers to any molecules that binds to or can be bound by another molecule.

As used herein, the term “detection region” refers to a discreet area on substrate that is designated for detection of an analyte (e.g., an organophosphate) in a sample.

As used herein, the term “immobilization” refers to the attachment or entrapment, either chemically or otherwise, of a material to another entity (e.g., a solid support) in a manner that restricts the movement of the material.

As used herein, the terms “material” and “materials” refer to, in their broadest sense, any composition of matter.

As used herein, the terms “field testing” refers to testing that occurs outside of a laboratory environment. Such testing can occur indoors or outdoors at, for example, a worksite, a place of business, public or private land, or in a vehicle.

As used herein, term “nanostructures” refers to microscopic structures, typically measured on a nanometer scale. Such structures include various three-dimensional assemblies, including, but not limited to, liposomes, films, multilayers, braided, lamellar, helical, tubular, and fiber-like shapes, and combinations thereof. Such structures can, in some embodiments, exist as solvated polymers in aggregate forms such as rods and coils. Such structures can also be formed from inorganic materials, such as prepared by the physical deposition of a gold film onto the surface of a solid, proteins immobilized on surfaces that have been mechanically rubbed, and polymeric materials that have been molded or imprinted with topography by using a silicon template prepared by electron beam lithography.

As used herein, the terms “self-assembling monomers” and “lipid monomers” refer to molecules that spontaneously associate to form molecular assemblies. In one sense, this can refer to surfactant molecules that associate to form surfactant molecular assemblies. The term “self-assembling monomers” includes single molecules (e.g., a single lipid molecule) and small molecular assemblies (e.g., polymerized lipids), whereby the individual small molecular assemblies can be further aggregated (e.g., assembled and polymerized) into larger molecular assemblies.

As used herein, the term “linker” or “spacer molecule” refers to material that links one entity to another. In one sense, a molecule or molecular group can be a linker that is covalent attached two or more other molecules (e.g., linking a ligand to a self-assembling monomer).

As used herein, the term “bond” refers to the linkage between atoms in molecules and between ions and molecules in crystals. The term “single bond” refers to a bond with two electrons occupying the bonding orbital. Single bonds between atoms in molecular notations are represented by a single line drawn between two atoms (e.g., C—C). The term “double bond” refers to a bond that shares two electron pairs. Double bonds are stronger than single bonds and are more reactive. The term “triple bond” refers to the sharing of three electron pairs. As used herein, the term “ene-yne” refers to alternating double and triple bonds. As used herein the terms “amine bond,” “thiol bond,” and “aldehyde bond” refer to any bond formed between an amine group (i.e., a chemical group derived from ammonia by replacement of one or more of its hydrogen atoms by hydrocarbon groups), a thiol group (i.e., sulfur analogs of alcohols), and an aldehyde group (i.e., the chemical group —CHO joined directly onto another carbon atom), respectively, and another atom or molecule.

As used herein, the term “covalent bond” refers to the linkage of two atoms by the sharing of two electrons, one contributed by each of the atoms.

As used herein, the term “spectrum” refers to the distribution of light energies arranged in order of wavelength.

As used the term “visible spectrum” refers to light radiation that contains wavelengths from approximately 360 nm to approximately 800 nm.

As used herein, the term “substrate” refers to a solid object or surface upon which another material is layered or attached. Solid supports include, but are not limited to, glass, metals, gels, and filter paper, among others.

As used herein, the terms “array” and “patterned array” refer to an arrangement of elements (i.e., entities) into a material or device. For example, combining several types of metal ions into an analyte-detecting device, would constitute an array.

As used herein, the term “in situ” refers to processes, events, objects, or information that are present or take place within the context of their natural environment.

As used herein, the term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

As used herein, the term “liquid crystal” refers to a thermodynamic stable phase characterized by anisotropy of properties without the existence of a three-dimensional crystal lattice, generally lying in the temperature range between the solid and isotropic liquid phase.

As used herein, the term “mesogen” refers compounds that form liquid crystals, including rodlike or disclike molecules which are components of liquid crystalline materials.

As used herein, “thermotropic liquid crystal” refers to liquid crystals which result from the melting of mesogenic solids due to an increase in temperature. Both pure substances and mixtures form thermotropic liquid crystals.

“Lyotropic,” as used herein, refers to molecules which form phases with orientational and/or positional order in a solvent. Lyotropic liquid crystals can be formed using amphiphilic molecules (e.g., sodium laurate, phosphatidylethanolamine, lecithin). The solvent can be water.

As used herein, the term “heterogenous surface” refers to a surface that orients liquid crystals in at least two separate planes or directions, such as across a gradient.

As used herein, “nematic” refers to liquid crystals in which the long axes of the molecules remain substantially parallel, but the positions of the centers of mass are randomly distributed. Nematic liquid crystals can be substantially oriented by a nearby surface.

“Chiral nematic,” as used herein refers to liquid crystals in which the mesogens are optically active. Instead of the director being held locally constant as is the case for nematics, the director rotates in a helical fashion throughout the sample. Chiral nematic crystals show a strong optical activity that is much higher than can be explained on the bases of the rotatory power of the individual mesogens. When light equal in wavelength to the pitch of the director impinges on the liquid crystal, the director acts like a diffraction grating, reflecting most and sometimes all of the light incident on it. If white light is incident on such a material, only one color of light is reflected and it is circularly polarized. This phenomenon is known as selective reflection and is responsible for the iridescent colors produced by chiral nematic crystals.

“Smectic,” as used herein refers to liquid crystals which are distinguished from “nematics” by the presence of a greater degree of positional order in addition to orientational order; the molecules spend more time in planes and layers than they do between these planes and layers. “Polar smectic” layers occur when the mesogens have permanent dipole moments. In the smectic A2 phase, for example, successive layers show anti ferroelectric order, with the direction of the permanent dipole alternating from layer to layer. If the molecule contains a permanent dipole moment transverse to the long molecular axis, then the chiral smectic phase is ferroelectric. A device utilizing this phase can be intrinsically bistable.

“Frustrated phases,” as used herein, refers to another class of phases formed by chiral molecules. These phases are not chiral, however, twist is introduced into the phase by an array of grain boundaries. A cubic lattice of defects (where the director is not defined) exist in a complicated, orientationally ordered twisted structure. The distance between these defects is hundreds of nanometers, so these phases reflect light just as crystals reflect x-rays.

“Discotic phases” are formed from molecules which are disc shaped rather than elongated. Usually these molecules have aromatic cores and six lateral substituents. If the molecules are chiral or a chiral dopant is added to a discotic liquid crystal, a chiral nematic discotic phase can form.

### DESCRIPTION OF THE INVENTION

The present invention relates to the field of detection of analytes, and in particular to detection of organophosphates using a liquid crystal assay format and a variety of analytes in stand-off detection formats utilizing liquid crystals as part of reporting system. Liquid crystal-based assay systems and devices (LC assays) are described in U.S. Pat. No. 6,284,197; WO 01/61357; WO 01/61325; WO 99/63329; Gupta et al., Science 279:2077-2080 (1998); Seung-Ryeol Kim, Rahul R. Shah, and Nicholas L. Abbott; Orientations of Liquid Crystals on Mechanically Rubbed Films of Bovine Serum Albumin: A Possible Substrate for Biomolecular Assays Based on Liquid Crystals, Analytical Chemistry; 2000; 72(19); 4646-4653; Justin J. Skaife and Nicholas L. Abbott; Quantitative Interpretation of the Optical Textures of Liquid Crystals Caused by Specific Binding of Immunoglobulins to Surface-Bound Antigens, Langmuir; 2000; 16(7); 3529-3536; Vinay K. Gupta and Nicholas L. Abbott; Using Droplets of Nematic Liquid Crystal To Probe the Microscopic and Mesoscopic Structure of Organic Surfaces, Langmuir; 1999; 15(21): 7213-7223; and Shah and Abbott, Principals for Measurement of Chemical Exposure Based on Recognition-Driven Anchoring Transitions in Liquid Crystals, Science 293:1296-99 (2001); all of which are incorporated herein by reference.

U.S. Pat. No. 6,284,197 and Shah and Abbott, supra, describe the detection of chemical molecules, including organophosphates, with a liquid crystal assay format. Surprisingly, it has now been discovered that liquid crystal assays can be tuned for the detection of particular organophosphates compounds and that the use of different metal ions in the assays can be lead to the identification of organophosphates through a particular “fingerprint” created by the organophosphates interaction with a variety metal ions. Furthermore, the liquid crystal assay devices of the present invention can be used to measure cumulative exposure to organophosphates.

In practice, prior art assessment of pesticide exposure has involved use of quantitative monitoring data (if available), or qualitative data (scenarios based on label use rates and assumptions on transport, transformation, and human behavior) to describe contact with and entry into the human body. In these assessments the “gold standard” is measurement of personal exposure. Personal (or point-of-contact) samplers document exposures as they occur by measuring the pesticide concentration at the point of contact between the person and the environmental medium. For example, current reference method pesticide samplers couple a pump and filters with a back up sorbant to collect pesticides mass (e.g., μg/m³) near the breathing zone, or skin patches made of cotton or other materials to measure dermal deposition (e.g., μg/cm²). The major strength of personal monitoring is that it measures exposure directly during the monitoring period, which typically is on the order of minutes, hours, or, at the most, days. The problems with these types of personal measurements is that they are costly and time consuming, require relatively expensive chemical analyses, can be burdensome for the study participants, and suitable monitoring devices are not available for all pesticides and pathways of interest (Adgate and Sexton 2001, Emerging Issues: Children's Exposure to Pesticides in Residential Settings. Handbook of Pesticide Toxicology, Second Edition. R. I. Krieger. San Diego, Academic Press. 1: 887-904). Because these problems are exacerbated in the case of children, personal monitoring has rarely been attempted in this subpopulation (Weaver et al., Approaches to environmental exposure assessment in children. Environ Health Perspect 1998: 106 Suppl 3: 827-32.1998).

While there is increasing concern about the effects pesticide exposure has on health, particularly for children in agricultural communities, and there is an increasing interest and need for expanded monitoring of personal exposure (for example, the planned The National Children's Study in which 100,000 children will be monitored for 20 years), the problems inherent in personal monitoring methods, particularly when applied to children, limit the number and nature of studies that can be done. According to a report on Human Exposure Assessment from the International Programme on Chemical Safety, “The principal limitation on the use of personal monitoring for exposure assessment is the availability of sample collection methods that are sensitive, easy to operate, able to provide sufficient time resolution, free from interferences and are cost effective.” (Macintosh and Spengler, Human Exposure Assessment. Environmental Health Criteria 214. World Health Organization, Geneva, 2000).

In quantifying exposure to OPs, it is important that the physical and chemical properties of the specific compound and formulation be examined. The measurement of OP pesticide exposure presents a special challenge because these compounds are semi-volatile. Semi-volatile organic compounds (SVOCs) have vapor pressures between 10⁻⁴ and 10⁻¹¹ atmospheres over the ambient temperature range, and can exist simultaneously in both gas and particle phases.

Methods that attempt to measure both vapor and particulate phases have been developed but can be associated with sampling artifacts that may underestimate or overestimate exposure. Underestimation may occur if deposited particles evaporate during and following sampling, which can happen if the vapor pressure of the material and the surface area of the particles are sufficiently high. Overestimation may occur if gas-phase SVOCs adsorb to the filter during sampling. The occurrence of either of these possibilities depends on the partitioning coefficient for the compound, the temperature, and the mass and types of particles present in the aerosol. Standard NIOSH methods for measurement of pesticides measure both the filter and the sorbent and therefore eliminate this artifact in the total concentration measurement.

In preferred embodiments, the devices of the present invention measure pesticides in the vapor phase, which lends itself to continuous monitoring without the need for extraction procedures. The present invention is not limited to any particular mechanism of action. Indeed, an understanding of the mechanism of action is not necessary to practice the present invention. Nevertheless, it is contemplated that the measurement of vapor phase provides an accurate determination of a major route of exposure and be reflective of other routes of pesticide exposure as the vapor phase is proportional to particulate and solids in the environment. The amount of pesticide in the vapor phase is expected to increase with increases in amounts of pesticide in other forms. The monitors also measure aerosol phase pesticides if these are collected and converted into the vapor phase.

Accordingly, the present invention provides improved substrates and devices for the detection of organophosphates. For convenience, the description of the present invention is divided into the following sections: I. Organophosphates; II. Recognition Moieties; III. Substrates; IV. Functionalization of Substrates; V. Mesogens; VI. Detection of Pathogens; VII. Kits.

I. Organophosphates

The present invention finds use in the detection of variety of organophosphates. In some embodiments, the organophosphates are those used as pesticides, including, but not limited to, Acephate (Orthene), Azinphos-ethyl, Azinphos-methyl (Guthion), Azinphos-methyl oxon, Bromophos-methyl, Carbophenothion (Trithion), Chlorfenvinphos (Supona), Chloropyrifos (Dursban/Lorsban), Chlorpyrifos-methyl, Chlorthiophos, Coumaphos (Co-Ral), Crotoxyphos (Ciodrin), Cyanophos, DEF (Butifos), Demeton (Systox), Demeton-Dialifor (Torak), Diazinon (O Analog), Diazinon (Spectracide), Dichlorvos-DDVP (Vapona), Dicrotophos (Bidrin), Dimethoate (Cygon), Dioxathion (Delnav), Disulfoton (Disyston), Disulfoton Sulfone, Edifenphos, EPN, Ethion (Nialate), Ethoprop (Mocap), Ethyl Parathion, Fenamiphos (Nemacur), Fenitrothion (Sumithion), Fensulfothion (Dasanit), Fenthion (Baytex), Fonofos (Dyfonate), Formothion, Heptenophos, Imidan (Phosmet), Isazophos (Triumph), Isofenphos (Amaze), Leptophos (Phosvel), Malaoxon, Malathion (Celthion), Merphos (Tribufos), Methamidophos (Monitor 4), Methidathion, Methyl Parathion (Metacide), Mevinphos (Phosdrin), Monocrotophos, Naled, Omethoate (Dimethoate O analog), Parathion (Alkron), Paroxon, Phorate (Thimet), Phorate-o, Phorate Sulfone, Phorate Sulfoxide, Phosalone, Phosphamidon (Dimecron), Piperophos, Pirimiphos-ethyl, Pirimiphos-methyl, Profenofos (Curacron), Propetamphos (Safrotin), Pyrazophos (Afgan), Quinalphos, Ronnel (Ectoral) (Fenchlorphos), Sulprofos (Bolstar), Terbufos (Counter), Tetrachlorvinphos (Gardona), Thionazin (Zinophos), and Triazophos (Hostathion). In some embodiments, the organophosphates are nerve agents (e.g., agents of war), including, but not limited to G agents (GD, soman; GB, sarin; and GA, tabun) and the V agents (VX).

II. Recognition Moieties

A variety of recognition moieties find use in the present invention. In preferred embodiments, the recognition moieties are immobilized on detection regions of the substrate (described in more detail below). In preferred embodiments, the recognition moieties for organophosphates include metal ions. The present invention is not limited to the use of any particular metal ion. Indeed, the use of a variety of metal ions is contemplated, including, but not limited to, Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺ and combinations thereof. In some embodiments, an organic layer is treated with ethanolic solutions of metal salts (containing the previously ions) to form the metal receptors on the surfaces of the organic layer. In particularly preferred embodiments, the metal ion interacts with one or more organophosphate compounds of interest (e.g., malathion, parathion or diazanon), but does not substantially interact (e.g., display a response of least 95% less or 99% less than that of the targeted organophosphate) with an interfering substance such as exhaust from internal combustion engines, kitchen odors, wood smoke, perfume, gasoline, diesel fuel, fertilizer, ammonia, baby lotion, hair spray, nail polisher (acetone), insecticides, cigarette smoke, NOₓ, CO, floor cleaners, furniture polish and household deodorizers.

III. Substrates

Substrates that are useful in practicing the present invention can be made of practically any physicochemically stable material. In a preferred embodiment, the substrate material is non-reactive towards the constituents of the mesogenic layer. The substrates can be either rigid or flexible and can be either optically transparent or optically opaque. The substrates can be electrical insulators, conductors or semiconductors. Further, the substrates can be substantially impermeable to liquids, vapors and/or gases or, alternatively, the substrates can be permeable to one or more of these classes of materials. Exemplary substrate materials include, but are not limited to, inorganic crystals, inorganic glasses, inorganic oxides, metals, organic polymers and combinations thereof. In preferred embodiments, the substrate comprises a planar gold coating that is not anisotropic. In some embodiments, the substrates have microchannels therein for the delivery of sample and/or other reagents to the substrate surface or detection regions thereon. The design and use of microchannels are described, for example, in U.S. Pat. Nos. 6,425,972, 6,418,968, 6,447,727, 6,432,720, 5,976,336, 5,882,465, 5,876,675, 6,186,660, 6,100,541, 6,379,974, 6,267,858, 6,251,343, 6,238,538, 6,182,733, 6,068,752, 6,429,025, 6,413,782, 6,274,089, 6,150,180, 6,046,056, 6,358,387, 6,321,791, 6,326,083, 6,171,067, and 6,167,910, all of which are incorporated herein by reference.

A. Inorganic Crystal and Glasses

In some embodiments of the present invention, inorganic crystals and inorganic glasses are utilized as substrate materials (e.g., LiF, NaF, NaCl, KBr, KI, CaF₂, MgF₂, HgF₂, BN, AsS₃, ZnS, Si₃N₄ and the like). The crystals and glasses can be prepared by art standard techniques (See, e.g., Goodman, C. H. L., Crystal Growth Theory and Techniques, Plenum Press, New York 1974). Alternatively, the crystals can be purchased commercially (e.g., Fischer Scientific). The crystals can be the sole component of the substrate or they can be coated with one or more additional substrate components. Thus, it is within the scope of the present invention to utilize crystals coated with, for example one or more metal films or a metal film and an organic polymer. Additionally, a crystal can constitute a portion of a substrate which contacts another portion of the substrate made of a different material, or a different physical form (e.g., a glass) of the same material. Other useful substrate configurations utilizing inorganic crystals and/or glasses will be apparent to those of skill in the art.

B. Inorganic Oxides In other embodiments of the present invention, inorganic oxides are utilized as the substrate. Inorganic oxides of use in the present invention include, for example, Cs₂O, Mg(OH)₂, TiO₂, ZrO₂, CeO₂, Y₂O₃, Cr₂O₃, Fe₂O₃, NiO, ZnO, Al₂O₃, SiO₂ (glass), quartz, In₂O₃, SO₂, PbO₂ and the like. The inorganic oxides can be utilized in a variety of physical forms such as films, supported powders, glasses, crystals and the like. A substrate can consist of a single inorganic oxide or a composite of more than one inorganic oxide. For example, a composite of inorganic oxides can have a layered structure (i.e., a second oxide deposited on a first oxide) or two or more oxides can be arranged in a contiguous non-layered structure. In addition, one or more oxides can be admixed as particles of various sizes and deposited on a support such as a glass or metal sheet. Further, a layer of one or more inorganic oxides can be intercalated between two other substrate layers (e.g., metal-oxide-metal, metal-oxide-crystal).

In some embodiments, the substrate is a rigid structure that is impermeable to liquids and gases. In this embodiment, the substrate consists of a glass plate onto which a metal, such as gold is layered by evaporative deposition. In a still further preferred embodiment, the substrate is a glass plate (SiO₂) onto which a first metal layer such as titanium or gold has been layered. A layer of a second metal (e.g., gold) is then layered on top of the first metal layer (e.g., titanium).

C. Metals

In still further embodiments of the present invention, metals are utilized as substrates. The metal can be used as a crystal, a sheet or a powder. The metal can be deposited onto a backing by any method known to those of skill in the art including, but not limited to, evaporative deposition, sputtering, electroless deposition, electrolytic deposition and adsorption or deposition of preform particles of the metal including metallic nanoparticles.

Any metal that is chemically inert towards the mesogenic layer will be useful as a substrate in the present invention. Metals that are reactive or interactive towards the mesogenic layer will also be useful in the present invention. Metals that are presently preferred as substrates include, but are not limited to, gold, silver, platinum, palladium, nickel and copper. In one embodiment, more than one metal is used. The more than one metal can be present as an alloy or they can be formed into a layered “sandwich” structure, or they can be laterally adjacent to one another. In a preferred embodiment, the metal used for the substrate is gold. In a particularly preferred embodiment the metal used is gold layered on titanium.

The metal layers can be either permeable or impermeable to materials such as liquids, solutions, vapors and gases.

D. Organic Polymers

In still other embodiments of the present invention, organic polymers are utilized as substrate materials. Organic polymers useful as substrates in the present invention include polymers that are permeable to gases, liquids and molecules in solution. Other useful polymers are those that are impermeable to one or more of these same classes of compounds.

Organic polymers that form useful substrates include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers and phenolic resins (See, Cognard, J. ALIGNMENT OF NEMATIC LIQUID CRYSTALS AND THEIR MIXTURES, in Mol. Cryst. Liq. Cryst. 1:1-74 (1982)). Presently preferred organic polymers include polydimethylsiloxane, polyethylene, polyacrylonitrile, cellulosic materials, polycarbonates and polyvinyl pyridinium.

In some embodiments, the substrate is permeable and it consists of a layer of gold, or gold over titanium, which is deposited on a polymeric membrane, or other material, that is permeable to liquids, vapors and/or gases. The liquids and gases can be pure compounds (e.g., chloroform, carbon monoxide) or they can be compounds which are dispersed in other molecules (e.g., aqueous protein solutions, herbicides in air, alcoholic solutions of small organic molecules). Useful permeable membranes include, but are not limited to, flexible cellulosic materials (e.g., regenerated cellulose dialysis membranes), rigid cellulosic materials (e.g., cellulose ester dialysis membranes), rigid polyvinylidene fluoride membranes, polydimethylsiloxane and track etched polycarbonate membranes.

In a further preferred embodiment, the layer of gold on the permeable membrane is itself permeable. In a still further preferred embodiment, the permeable gold layer has a thickness of about 70 Angstroms or less.

In those embodiments wherein the permeability of the substrate is not a concern and a layer of a metal film is used, the film can be as thick as is necessary for a particular application. For example, if the film is used as an electrode, the film can be thicker than in an embodiment in which it is necessary for the film to be transparent or semi-transparent to light.

Thus, in a preferred embodiment, the film is of a thickness of from about 0.01 nanometer to about 1 micrometer. In a further preferred embodiment, the film is of a thickness of from about 5 nanometers to about 100 nanometers. In yet a further preferred embodiment, the film is of a thickness of from about 10 nanometers to about 50 nanometers.

IV. Functionalization of Substrates

In some embodiments, the surface of the substrate is functionalized so that a recognition moiety (e.g., a metal ion) can be immobilized on the surface of the substrate, thereby forming a detection region. In some embodiments, a plurality of detection regions are formed on the surface of the substrate. In some embodiments, the same metal ion is provided on two or more of the plurality of detection regions, while in other embodiments, at least two different metal ions are immobilized on one or more of the plurality of detection regions. In some embodiments, the metal ions are arrayed in discreet detection regions on the substrate surfaces by the methods described in more detail below.

A. Organic Monolayers

In some embodiments, the surface of the substrate is first functionalized by forming an organic layer, such as a self-assembled monolayer (SAM) on the substrate surface. Self-assembled monolayers are generally depicted as an assembly of organized, closely packed linear molecules. There are two widely-used methods to deposit molecular monolayers on solid substrates: Langmuir-Blodgett transfer and self-assembly. Additional methods include techniques such as depositing a vapor of the monolayer precursor onto a substrate surface and the layer-by-layer deposition of polymers and polyelectrolytes from solution (Ladam et al., Protein Adsorption onto Auto-Assembled Polyelectrolyte Films, Langmuir; 2001; 17(3); 878-882). In preferred embodiments, the organic layer is formed from 11-mercaptoundecanoic acid, 4-aminothiophenol, or mercaptobenzoic acid.

It will be recognized that the composition of a layer of a SAM useful in the present invention can be varied over a wide range of compound structures and molar ratios. In one embodiment, the SAM is formed from only one compound. In some preferred embodiments, the SAM is formed from two or more components. In other preferred embodiments, when two or more components are used, one component is a long-chain hydrocarbon having a chain length of between 10 and 25 carbons and a second component is a short-chain hydrocarbon having a chain length of between 1 and 9 carbon atoms. In particularly preferred embodiments, the SAM is formed from CH₃(CH₂)₁₅SH and CH₃(CH₂)₄SH or CH₃(CH₂)₁₅SH and CH₃(CH₂)₉SH. In any of the above described embodiments, the carbon chains can be functionalized at the co-terminus (e.g., NH₂, COOH, OH, CN), at internal positions of the chain (e.g., aza, oxa, thia) or at both the co-terminus and internal positions of the chain.

B. Functionalized SAMs

The discussion that follows focuses on the attachment of a reactive SAM component to the substrate surface. This focus is for convenience only and one of skill in the art will understand that the discussion is equally applicable to embodiments in which the SAM component-recognition moiety is preformed prior to its attachment to the substrate. As used herein, “reactive SAM components” refers to components that have a functional group available for reaction with a recognition moiety or other species following the attachment of the component to the substrate.

Currently favored classes of reactions available with reactive SAM components are those that proceed under relatively mild conditions. These include, but are not limited to nucleophilic substitutions (e.g., reactions of amines and alcohols with acyl halides), electrophilic substitutions (e.g., enamine reactions) and additions to carbon-carbon and carbon-heteroatom multiple bonds (e.g., Michael reaction, Diels-Alder addition). These and other useful reactions are discussed in March, ADVANCED ORGANIC CHEMISTRY, Third Ed., John Wiley & Sons, New York, 1985.

In some embodiments of the present invention, a substrate's surface is functionalized with SAM, components and other species by covalently binding a reactive SAM component to the substrate surface in such a way as to derivatize the substrate surface with a plurality of available reactive functional groups. Reactive groups which can be used in practicing the present invention include, for example, amines, hydroxyl groups, carboxylic acids, carboxylic acid derivatives, alkenes, sulfhydryls, siloxanes, etc.

A wide variety of reaction types are available for the functionalization of a substrate surface. For example, substrates constructed of a plastic such as polypropylene, can be surface derivatized by chromic acid oxidation, and subsequently converted to hydroxylated or aminomethylated surfaces. Substrates made from highly crosslinked divinylbenzene can be surface derivatized by chloromethylation and subsequent functional group manipulation. Additionally, functionalized substrates can be made from etched, reduced polytetrafluoroethylene.

When the substrates are constructed of a siliaceous material such as glass, the surface can be derivatized by reacting the surface Si—OH, SiO—H, and/or Si—Si groups with a functionalizing reagent. When the substrate is made of a metal film, the surface can be derivatized with a material displaying avidity for that metal.

In a preferred embodiment, wherein the substrates are made from glass, the covalent bonding of the reactive group to the glass surface is achieved by conversion of groups on the substrate's surface by a silicon modifying reagent such as:
(RO)₃—Si—R¹—X¹ (1)
where R is an alkyl group, such as methyl or ethyl, R¹ is a linking group between silicon and X and X is a reactive group or a protected reactive group. The reactive group can also be a recognition moiety as discussed below. Silane derivatives having halogens or other leaving groups beside the displayed alkoxy groups are also useful in the present invention.

A number of siloxane functionalizing reagents can be used, for example:
1. Hydroxyalkyl siloxanes (Silylate surface, functionalize with diborane, and H₂0₂ to oxidize the alcohol)
   a. allyl trichlorosilane→→3-hydroxypropyl
   b. 7-oct-1-enyl trichlorosilane→→8-hydroxyoctyl
2. Diol (dihydroxyalkyl) siloxanes (silylate surface and hydrolyze to diol)
   a. (glycidyl trimethoxysilane→→(2,3-dihydroxypropyloxy)propyl
3. Aminoalkyl siloxanes (amines requiring no intermediate functionalizing step).
   a. 3-aminopropyl trimethoxysilane→aminopropyl
4. Dimeric secondary aminoalkyl siloxanes
   a. bis (3-trimethoxysilylpropyl)amine→bis(silyloxylpropyl)amine.

It will be apparent to those of skill in the art that an array of similarly useful functionalizing chemistries are available when SAM components other than siloxanes are used. Thus, for example similarly functionalized alkyl thiols can be attached to metal films and subsequently reacted to produce the functional groups such as those exemplified above.

In another preferred embodiment, the substrate is at least partially a metal film, such as a gold film, and the reactive group is tethered to the metal surface by an agent displaying avidity for that surface. In a presently preferred embodiment, the substrate is at least partially a gold film and the group which reacts with the metal surface comprises a thiol, sulfide or disulfide such as:
Y—S—R²—X² (2)
R² is a linking group between sulfur and X² and X² is a reactive group or a protected reactive group. X² can also be a recognition moiety as discussed below. Y is a member selected from the group consisting of H, R³ and R³—S—, wherein R² and R³ are independently selected. When R² and R³ are the same, symmetrical sulfides and disulfides result, and when they are different, asymmetrical sulfides and disulfides result.

A large number of functionalized thiols, sulfides and disulfides are commercially available (Aldrich Chemical Co., St. Louis). Additionally, those of skill in the art have available to them a manifold of synthetic routes with which to produce additional such molecules. For example, amine-functionalized thiols can be produced from the corresponding halo-amines, halo-carboxylic acids, etc. by reaction of these halo precursors with sodium sulfhydride. See, e.g., Reid, ORGANIC CHEMISTRY of BIVALENT SULFUR, VOL 1, pp. 21-29, 32-35, vol. 5, pp. 27-34, Chemical Publishing Co., New York, 1.958, 1963. Additionally, functionalized sulfides can be prepared via alkylthio-de-halogenation with a mercaptan salt (See, Reid, ORGANIC CHEMISTRY OF BIVALENT SULFUR, vol. 2, pp. 16-21, 24-29, vol. 3, pp. 11-14, Chemical Publishing Co., New York, 1960). Other methods for producing compounds useful in practicing the present invention will be apparent to those of skill in the art.

In another preferred embodiment, the functionalizing reagent provides for more than one reactive group per each reagent molecule. Using reagents such as Compound 3, below, each reactive site on the substrate surface is, in essence, “amplified” to two or more functional groups:
(RO)₃—Si—R²—(X²)ₙ (3)
where R is an alkyl group, such as methyl, R² is a linking group between silicon and X², X² is a reactive group or a protected reactive group and n is an integer between 2 and 50, and more preferably between 2 and 20.

Similar amplifying molecules are also of use in those embodiments wherein the substrate is at least partially a metal film. In these embodiments the group which reacts with the metal surface comprises a thiol, sulfide or disulfide such as in Formula (4):
Y—S—R²—(X²)ₙ (4)
As discussed above, R² is a linking group between sulfur and X² and X² is a reactive group or a protected reactive group. X² can also be a recognition moiety. Y is a member selected from the group consisting of H, R³ and R³—S—, wherein R and R³ are independently selected.

R groups of use for R¹, R² and R³ in the above described embodiments of the present invention include, but are not limited to, alkyl, substituted alkyl, aryl, arylalkyl, substituted aryl, substituted arylalkyl, acyl, halogen, hydroxy, amino, alkylamino, acylamino, alkoxy, acyloxy, aryloxy, aryloxyalkyl, mercapto, saturated cyclic hydrocarbon, unsaturated cyclic hydrocarbon, heteroaryl, heteroarylalkyl, substituted heteroaryl, substituted heteroarylalkyl, heterocyclic, substituted heterocyclic and heterocyclicalkyl groups.

In each of Formulae 1-4, above, each of R¹, R² and R³ are either stable or they can be cleaved by chemical or photochemical reactions. For example, R groups comprising ester or disulfide bonds can be cleaved by hydrolysis and reduction, respectively. Also within the scope of the present invention is the use of R groups which are cleaved by light such as, for example, nitrobenzyl derivatives, phenacyl groups, benzoin esters, etc. Other such cleaveable groups are well-known to those of skill in the art.

In another preferred embodiment, the organosulfur compound is partially or entirely halogenated. An example of compounds useful in this embodiment include:
X¹Q₂C(CQ¹₂)ₘZ¹(CQ²₂)ₙSH (5)
wherein, X¹ is a member selected from the group consisting of H, halogen reactive groups and protected reactive groups. Reactive groups can also be recognition moieties as discussed below. Q, Q¹ and Q² are independently members selected from the group consisting of H and halogen. Z¹ is a member selected from the group consisting of —CQ₂—, —CQ¹₂—, —CQ²₂—, —O—, —S—, NR⁴—, —C(O)NR⁴ and R⁴NC(O0-, in which R⁴ is a member selected from the group consisting of H, alkyl, substituted alkyl, aryl, substituted aryl, heteroaryl and heterocyclic groups and m and n are independently a number between 0 and 40.

In yet another preferred embodiment, the organic layer comprises a compound according to Formula 5 above, in which Q, Q¹ and Q² are independently members selected from the group consisting of H and fluorine. In a still further preferred embodiment, the organic layer comprises compounds having a structure according to Formulae (6) and (7):
CF₃(CF₂)ₘZ¹(CH₂)ₙSH (6)
CF₃(CF₂)ₒZ²(CH₂)ₚSH (7)
wherein, Z¹ and Z² are members independently selected from the group consisting of —CH₂—, —O—, —S—, NR⁴, —C(O)NR⁴ and R⁴NC(O)— in which R⁴ is a member selected from the group consisting of H, alkyl, substituted alkyl, aryl, substituted aryl, heteroaryl and heterocyclic groups. In a presently preferred embodiment, the Z groups of adjacent molecules participate in either an attractive (e.g., hydrogen bonding) or repulsive (e.g., van der Waals) interaction.

In Formula 7, m is a number between 0 and 40, n is a number between 0 and 40, o is a number between 0 and 40 and p is a number between 0 and 40.

In a further preferred embodiment, the compounds of Formulae 6 and 7 are used in conjunction with an organosulfur compound, either halogentated or unhalogenated, that bears a recognition moiety.

When the organic layer is formed from a halogenated organosulfur compound, the organic layer can comprise a single halogenated compound or more than one halogenated compound having different structures. Additionally, these layers can comprise a non-halogenated organosulfur compound.

The reactive functional groups (X¹ and X²) are, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups which can be converted to disulfides or reacted with acyl halides;

(h) amine or sulfhydryl groups which can be, for example, acylated or alkylated;

(i) alkenes which can undergo, for example, cycloadditions, acylation, Michael addition, etc; and

(j) epoxides which can react with, for example, amines and hydroxyl compounds.

The reactive moieties can also be recognition moieties. The nature of these groups is discussed in greater detail below.

The reactive functional groups can be chosen such that they do not participate in, or interfere with, the reaction controlling the attachment of the functionalized SAM component onto the substrate's surface. Alternatively, the reactive functional group can be protected from participating in the reaction by the presence of a protecting group. Those of skill in the art will understand how to protect a particular functional group from interfering with a chosen set of reaction conditions. For examples of useful protecting groups, see Greene et al., PROTECTIVE GROUPS IN ORGANIC SYNTHESIS, John Wiley & Sons, New York, 1991.

When two or more structurally distinct moieties are used as components of the SAMs, the components can be contacted with the substrate as a mixture of SAM components or, alternatively, the components can be added individually. In those embodiments in which the SAM components are added as a mixture, the mole ratio of a mixture of the components in solution results in the same ratio in the mixed SAM. Depending on the manner in which the SAM is assembled, the two components do not phase segregate into islands (See, Bain and Whitesides, J. Am. Chem. Soc. 111:7164 (1989)). This feature of SAMs can be used to immobilize recognition moieties or bulky modifying groups in such a manner that certain interactions, such as steric hindrance, between these molecules is minimized.

The individual components of the SAMs can also be bound to the substrate in a sequential manner. Thus, in one embodiment, a first SAM component is attached to the substrate's surface by “underlabeling” the surface functional groups with less than a stoichiometric equivalent of the first component. The first component can be a SAM component liked to a terminal reactive group or recognition group, a spacer arm or a monovalent moiety. Subsequently, the second component is contacted with the substrate. This second component can either be added in stoichiometric equivalence, stoichiometric excess or can again be used to underlabel to leave sites open for a third component.

In some preferred embodiments, metal ions are attached to the organic layer by treating the organic layer with an ethanolic solution of a salt of the desired metal ions. Examples, of such metal ion salts include, but are not limited to Aluminum perchlorate, Silver perchlorate, Barium perchlorate, Cadmium perchlorate, Cerium perchlorate, Cobalt perchlorate, Chromium perchlorate, Copper perchlorate, Europium perchlorate, Iron(II) perchlorate, Iron (III) perchlorate, Gallium perchlorate, Indium perchlorate, Maganese perchlorate, Nickel perchlorate, Lead perchlorate, Praseodynium perchlorate, and Zinc perchlorate

C. Arrays

In some embodiments, the recognition moieties (e.g., metal ions) are arrayed on the substrates using stamping, microcontact printing, or ink-jet printing. In still further embodiments, recognition moieties are spotted onto a suitable substrate. Such spotting can be done by hand with a capillary tube or micropipette, or by an automated spotting apparatus such as those available from Affymetrix and Gilson (See e.g., U.S. Pat. Nos. 5,601,980; 6,242,266; 6,040,193; and 5,700,637; each of which is incorporated herein by reference).

V. Mesogens

Any compound or mixture of compounds that forms a mesogenic layer can be used in conjunction with the present invention. The mesogens can form thermotropic or lyotropic liquid crystals. Both the thermotropic and lyotropic liquid crystals can exist in a number of forms including nematic, chiral nematic, smectic, polar smectic, chiral smectic, frustrated phases and discotic phases.

TABLE 1
Molecular structure of mesogens suitable for use in Liquid Crystal Assay Devices

| Mesogen | Structure |
|---|---|
| Anisaldazine | 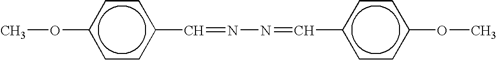 |
| NCB | 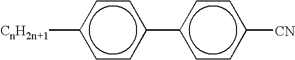 |
| CBOOA | 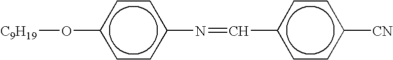 |
| Comp A | 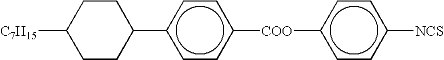 |
| Comp B | 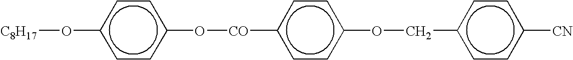 |
| DB₇NO₂ | 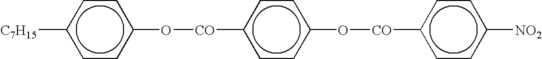 |
| DOBAMBC | 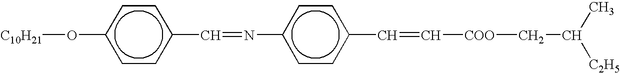 |
| nOm | 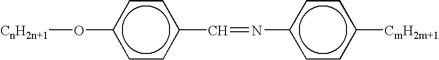 |
| n = 1, m = 4: MBBA | |
| n = 2, m = 4: EBBA | |
| nOBA | 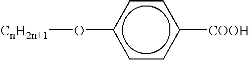 |
| n = 8: OOBA | |
| n = 9: NOBA | |
| nmOBC | 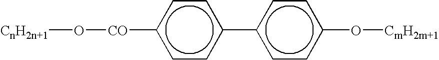 |
| nOCB | 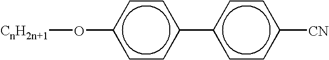 |
| nOSI | 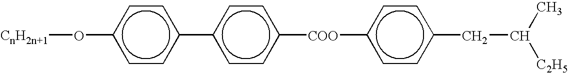 |
| 98P | 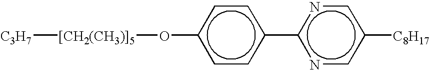 |
| PAA | 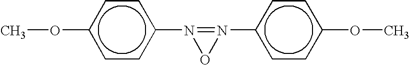 |
| PYP906 | 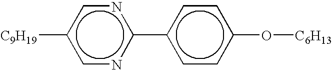 |
| n̅Sm | 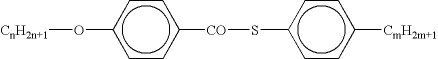 |

Presently preferred mesogens are displayed in Table 1. In a particularly preferred embodiment, the mesogen is a member selected from the group consisting of E7, 5CB (4-pentyl-4′-cyanobiphenyl), MLC, and 8CB (4-cyano-4′octylbiphenyl) and combinations thereof.

The mesogenic layer can be a substantially pure compound, or it can contain other compounds that enhance or alter characteristics of the mesogen. Thus, in one preferred embodiment, the mesogenic layer further comprises a second compound, for example an alkane, which expands the temperature range over which the nematic and isotropic phases exist. Use of devices having mesogenic layers of this composition allows for detection of the analyte recognition moiety interaction over a greater temperature range.

In some preferred embodiments, the mesogenic layer further comprises a dichroic dye or fluorescent compound. Examples of dichroic dyes and fluorescent compounds useful in the present invention include, but are not limited to, azobenzene, BTBP, polyazo compounds, anthraquinone, perylene dyes, and the like. In particularly preferred embodiments, a dichroic dye of fluorescent compound is selected that complements the orientation dependence of the liquid crystal so that polarized light is not required to read the assay. In some preferred embodiments, if the absorbance of the liquid crystal is in the visible range, then changes in orientation can be observed using ambient light without crossed polars. In other preferred embodiments, the dichroic dye or fluorescent compound is used in combination with a fluorimeter and the changes in fluorescence are used to detect changes in orientation of the liquid crystal.

VI. Detection of Organophosphates

The present invention provides methods and devices for the detection of organophosphates in a sample. The device of the present invention can be of any configuration which allows for the contact of a mesogenic layer with an organic layer or inorganic layer (e.g., metal, metal salt or metal oxide) decorated with a recognition moiety (e.g., metal ion). The only limitations on size and shape are those that arise from the situation in which the device is used or the purpose for which it is intended. In some embodiments, the devices comprise optical cells in which a first functionalized substrate is arranged opposite from a second substrate that may or may not be functionalized so as to form a chamber into which a liquid crystal can be introduced. In other embodiments, a single substrate that is open to the environment on one surface is utilized. The device can be planar or non-planar. Furthermore, it is within the scope of the present invention to use any number of polarizers, lenses, filters, lights, and the like to practice the present invention.

The present invention is not limited to any particular mechanism of action. Indeed, an understanding of the mechanism of action is not necessary to practice the present invention. Nevertheless, it is contemplated that the mesogens forming the liquid crystal of the devices of the present invention have an affinity for metal ions displayed on the organic layer. This affinity causes homeotropic ordering of the liquid crystal. Particular organophosphate analytes have higher affinities for particular metal ions than the mesogens. The devices of the present invention are designed so that when organophosphates are present in a sample, the organophosphates can enter detection regions of the device where metal ions are arrayed and disrupt the interaction of the mesogens with the metal ions by displacing the mesogens. This disruption creates an area of disorder in the liquid crystal (i.e., an unordered region as opposed to a homeotropically ordered region) that can be detected in a variety of ways.

Accordingly, in some embodiments, the present invention provides substrates comprising at least one detection region comprising a recognition moiety (e.g., a metal ion) that binds to or otherwise interacts with an organophosphate. In preferred embodiments, the detection regions are discreet and created by arraying at least one recognition moiety on the surface of the substrate. In preferred embodiments, the recognition moiety is immobilized on the substrate or the organic layer as described in detail above. In some embodiments, a plurality of metal ions are arrayed on the surface of the substrate so that multiplexed assays for a variety of organophosphates can be conducted or so that different interactions with a variety of metal ions can be used as a signature for a particular organophosphate. In some preferred embodiments, a stamp is used to transfer the metal salt or ion to the detection region. In some particularly preferred embodiments, the stamp is a PDMS stamp.

In some embodiments, a second substrate is provided which is configured opposite the first substrate so that a cell is formed. In some embodiments, the second substrate is also arrayed with recognition moieties, while in other embodiments, the second substrate is free of recognition moieties. In some preferred embodiments, the recognition moieties are arrayed on the first and second substrates so that when the first and second substrates are placed opposite each other the arrays match to form discreet detection regions.

In some embodiments, the cell that is formed by the first and second substrates includes a space between the first and second substrates. In some embodiments, the space is formed by placing a spacer between the first and second substrates. In some embodiments, the space is then filled with the desired liquid crystal. In still other embodiments, the substrates are arranged so that a sample can interact with or enter into the detection regions. In some embodiments, the substrates are fixed (e.g., permanently or removeably) to one another. The present invention is not limited to any particular mode of fixation. Indeed, a variety of modes of fixation are contemplated. In some embodiments, the substrates are fixed to one another via adhesive tape. In preferred embodiments, the adhesive tape is 8141 pressure sensitive adhesive (3M, Minneapolis, Minn.). In other embodiments, the substrates are fixed to one another via a UV curable adhesive. In some preferred embodiments, the UV curable adhesive is PHOTOLEC.® A704 or A720 (Sekisui, Hong Kong). In some embodiments, glass spacer rods are utilized with the UV curable adhesive to provide spacing between the two substrates. In some embodiments, the glass spacer rods range from about 5 μM to about 100 μM, preferably about 25 μM. It has been found that UV curable adhesives are preferable as in some instances the adhesive tape reacts with the liquid crystal.

In further embodiments, the substrates are arranged in a housing. The housing can comprise any suitable material, and is preferably made of plastic. In preferred embodiments, the housing is sealed to the environment except for an opening adjacent to the detection region or regions. The opening preferably allows diffusion of air to the detection region. In some embodiments, the opening is covered with a filter material that allows diffusion of air to the detection region, but does not allow entry of particulate matter such as dust, dirt, and insects into the detection region. In some embodiments, the filter is an aerosol filter that substantially prevents the introduction of aerosols into the detection region, but allows an analyte such an OP in vapor form to enter the detection region. In still more preferred embodiments, the devices comprise two or more filters positioned so as to allow air-exchange though the device, and in particular, through the detection region. For example, the filters can be arranged at either end of the detection region. In further embodiments, the housing is moveable between an exposure mode mode and a reading mode. In the exposure mode, the detection regions are exposed to the environment, while in the reading mode, exposure to the environment is substantially or completely eliminated. It is envisioned that after the device has been exposed to the environment, the housing can be moved to the reading mode to prevent further exposure to the environment prior to readout.

In still further embodiments, the devices of the present invention comprise a unique identifier. In some embodiments, the unique identifier is a bar code. In other embodiments, the unique identifier is an RFID chip. It is contemplated that the unique identifier can provide information such as a serial number, user identification, source identification, and the like.

In use, the device is preferably placed in area where organophosphates are suspected of being present or is attached to a person as personal monitor. The device is allowed to remain in place for a period of time (the exposure period, e.g., from 1 day to four weeks).

Following the exposure period, the cell is assayed for whether a change in the liquid crystal has occurred over one or more of the detection regions. Although many changes in the mesogenic layer can be detected by visual observation under ambient light, any means for detecting the change in the mesogenic layer can be incorporated into, or used in conjunction with, the device. Thus, it is within the scope of the present invention to use lights, microscopes, spectrometry, electrical techniques and the like to aid in the detection of a change in the mesogenic layer. In some embodiments, the presence of organophosphates is detected by a change in the color and texture of the liquid crystal.

Accordingly, in those embodiments utilizing light in the visible region of the spectrum, the light can be used to simply illuminate details of the mesogenic layer. Alternatively, the light can be passed through the mesogenic layer and the amount of light transmitted, absorbed or reflected can be measured. The device can utilize a backlighting device such as that described in U.S. Pat. No. 5,739,879, incorporated herein by reference. Light in the ultraviolet and infrared regions is also of use in the present invention. In other embodiments, the device, and in particular the detection region, is illuminated with monochromatic light source (e.g., 660 nm LEDs). In some embodiments, the cell is placed in between cross-polar lenses and light is passed though the lenses and the cell. In still other embodiments, the detection region is masked off from the rest of the device by a template or mask that is placed over the device.

The devices of the present invention are useful for measuring cumulative exposure to organophosphates. In some embodiments, cumulative exposure is assayed by determining the advancement of a wavefront in the detection region. It is contemplated that the wavefront advances from opening associated with the detection region. The distance of advancement correlates to the degree of exposure to organophosphates and is thus quantitative. In particular, it is contemplated that the rate of progress of the wavefront into the detection region depends on the concentration of organophosphate to which the device is exposed. In preferred embodiments, the front movement in millimeters is plotted against elapsed time in hours. The resulting plot obeys a linear fit (preferably with a coefficient of correlation of greater than 0.95) characteristic the concentration of organophosphate in the sample (e.g., local atmosphere). In some preferred embodiments, wavefront advancement is measured capturing a digital image of the detection region and determining the area and length of the wavefront from the opening in pixels. In some preferred embodiments, the image is analyzed with a program such as Scion Image (NIH Freeware). The pixels can then be converted into millimeters if necessary. In other embodiments, the image is analyzed by converting the image with a % white command so that the area in which the liquid crystal has been disrupted by the organophosphate appears white. The degree of advancement of the wavefront can be determined by measuring pixel intensity and determining where image drop-off from high intensity (white) to low intensity (black).

The devices of the present invention can also be used to identify particular organophosphates. In some embodiments, the detection region of the device comprises an array of at least two different metal ions. The pattern of response to the at least two different metal ions can be used to identify particular compounds.

VIII. Stand-Off Detection

In other embodiments, the present invention provides methods and devices for use in the detection of analyte from a remote distance (i.e., stand-off detection). It is contemplated that such embodiments will be especially useful for determining the presence of an analyte over a large area or environment, and in particular whether an analyte is present in the atmosphere. Thus, in some preferred embodiments, liquid crystal assay devices are provided that can be dispersed into the atmosphere and probed from a remote distance, for example, from about greater than 10 meters, 100 meters, 1 kilometer or 10 kilometers up to about 1000 kilometers or more in the case of a satellite. The devices may be configured to detect organophosphates as described above, or a number of additional analytes as described in more detail below.

In some embodiments, the devices of the present invention, which are described in more detail below, comprise at least one surface displaying a recognition moiety. In preferred embodiments, the devices comprise a second surface opposed to the first surface so as to create a chamber. In preferred embodiments, the chamber contains a liquid crystal. In some embodiments, the second surface also displays a recognition moiety. A variety of surfaces may be utilized, including those described in detail above. In some preferred embodiments, the first surface is gold and the second surface is nanoporous gold. In other embodiments, the surface is the surface of pore in porous silicon and the liquid crystal fills the pore.

In some embodiments, the devices are probed from a remote distance by irradiating the devices with electromagnetic radiation. The present invention is not limited to any particular mechanism of action. Indeed, an understanding of the mechanism of action of the present invention is not necessary to practice the present invention. Nevertheless, it is contemplated that the interaction between the functionalized nanostructures impregnated with liquid crystals (LCs) and electromagnetic radiation changes when the targeted analyte binds to the recognition moiety and induces an orientational transition in the liquid crystals. In some embodiments, these devices are small enough (e.g., 1-500 microns; preferably 1-100 microns) so that they tumbling in air. In other embodiments, the devices are slightly heavier so that they settle on the ground. It is contemplated that the change in liquid crystal orientation in the devices manifests as a change in the electromagnetic spectrum (wavelength or intensity) of the probing radiation.

It is further contemplated that the nature of the interaction between the nanostructures and the electromagnetic radiation depends strongly on the dielectric environment of the nanostructures. When the analyte binds to the surface of the nanostructures it causes a negligibly small change in the dielectric properties, which is extremely difficult to detect. However, when the functionalized nanostructures are impregnated with LCs, binding of the analyte to the receptor induces orientational transition of LCs that propagates throughout the nanostructures. This causes a large change in the dielectric property of the nanostructures and hence significant change in the probing electromagnetic spectrum.

Accordingly, in some embodiments, small pieces of the transducing elements (e.g., the devices of the present invention) with nanostructures are dispersed into air using airborne conveyance such as airplanes, helicopters, balloons, rockets, etc. Depending upon the size of the particles used and the preference of reading, these nanostructured materials tumble in air or settle in the field. In preferred embodiments, these transducing elements are interrogated from a remote location using electromagnetic radiation via a satellite, plane, or other mobile radiation source. The transmitter sends electromagnetic radiation on a regular basis and a detector collects the response sent back from these elements. As the target analyte binds to the receptor there is a change in the probing electromagnetic spectrum. This change can be probed in a number of different ways that can be divided in to three broad categories based on different mechanisms involved. In some embodiments, electromagnetic radiation is reflected back and the reflected spectrum changes when the target analyte binds to the nanostructures. In other embodiments, the device absorbs the incident radiation and re-radiates electromagnetic radiation at different wavelength. When the analyte binds to the receptor the radiated spectrum changes. In other embodiments, the device diffracts the electromagnetic radiation in specific directions. A property of the diffracted spectrum changes as the target analyte binds to the receptor. In some embodiments, elements that are configured to detect different analytes are dispersed together to provide a multiplexed stand-off assay. In these embodiments, the spectrum emitted by a first element configured to detect a first analyte is preferably different from the emission spectrum of a second element configured to detect a second analyte.

The stand off detection methods of the present invention are useful for the detection of a variety of analytes, including, but not limited to, biomolecules including polypeptides (e.g., proteins), toxins, polynucleotides (e.g., RNA and DNA), carbohydrates, viruses, mycoplasmas, fungi, bacteria, and protozoa, especially Class A agents such as Variola major (smallpox), Bacillus anthracis (anthrax), Yersinia pestis (plague), Clostridium botulinum (botulism), Francisella tularensis (tularemia), Arenaviruses (Arenaviridae), Ebola hemorrhagic fever virus, Marburg hemorrhagic fever, Lassa fever virus, Junin and related viruses (Argentinian hemorrhagic fever virus, Bolivian hemorrhagic fever virus, Brazilian hemorrhagic fever virus, Venezuelan hemorrhagic fever virus), Dengue hemorrhagic fever virus, and toxins such as botulinum and Trichothecene (T2) mycotoxins; Class B agents such as Coxiella burnetti (Q fever), Brucella sp. (brucellosis), Burkholderia mallei (glanders), Salmonella sp., Shigella dysenteria, Escherichia coli strain O 157:H7, Cryptosporidium parvum, Alphaviruses (Togaviridae family) such as Venezuelan equine encephalitis virus, Eastern equine encephalitis virus, Western equine encephalitis virus, and toxins such as ricin toxin, epsilin toxin from Clostridium perfigens, and Staphylococcus enterotoxin B; and Class C agents such as mutlidrug resistant tuberculosis, Nipah virus, Hantaviruses, Tick-borne hemorrhagoc fever viruses, Tick-borne encephalitis viruses, and Yellow fever virus. Other analytes include, but are not limited to, acids, bases, organic ions, inorganic ions, pharmaceuticals, herbicides, pesticides, chemical warfare agents, and noxious gases. These agents can be present as components in mixtures of structurally unrelated compounds, racemic mixtures of stereoisomers, non-racemic mixtures of stereoisomers, mixtures of diastereomers, mixtures of positional isomers or as pure compounds. Within the scope of the invention is a device and a method to detect a particular analyte of interest without interference from other substances within a mixture. Examples of organophosphate analytes include, but are not limited to the organophosphates used as pesticides, such as, Acephate (Orthene), Azinphos-ethyl, Azinphos-methyl (Guthion), Azinphos-methyl oxon, Bromophos-methyl, Carbophenothion (Trithion), Chlorfenvinphos (Supona), Chloropyrifos (Dursban/Lorsban), Chlorpyrifos-methyl, Chlorthiophos, Coumaphos (Co-Ral), Crotoxyphos (Ciodrin), Cyanophos, DEF (Butifos), Demeton (Systox), Demeton-Dialifor (Torak), Diazinon (O Analog), Diazinon (Spectracide), Dichlorvos-DDVP (Vapona), Dicrotophos (Bidrin), Dimethoate (Cygon), Dioxathion (Delnav), Disulfoton (Disyston), Disulfoton Sulfone, Edifenphos, EPN, Ethion (Nialate), Ethoprop (Mocap), Ethyl Parathion, Fenamiphos (Nemacur), Fenitrothion (Sumithion), Fensulfothion (Dasanit), Fenthion (Baytex), Fonofos (Dyfonate), Formothion, Heptenophos, Imidan (Phosmet), Isazophos (Triumph), Isofenphos (Amaze), Leptophos (Phosvel), Malaoxon, Malathion (Celthion), Merphos (Tribufos), Methamidophos (Monitor 4), Methidathion, Methyl Parathion (Metacide), Mevinphos (Phosdrin), Monocrotophos, Naled, Omethoate (Dimethoate O analog), Parathion (Alkron), Paroxon, Phorate (Thimet), Phorate-o, Phorate Sulfone, Phorate Sulfoxide, Phosalone, Phosphamidon (Dimecron), Piperophos, Pirimiphos-ethyl, Pirimiphos-methyl, Profenofos (Curacron), Propetamphos (Safrotin), Pyrazophos (Afgan), Quinalphos, Ronnel (Ectoral) (Fenchlorphos), Sulprofos (Bolstar), Terbufos (Counter), Tetrachlorvinphos (Gardona), Thionazin (Zinophos), and Triazophos (Hostathion) and nerve agents (e.g., agents of war), including, but not limited to G agents (GD, soman; GB, sarin; and GA, tabun), and V agents (VX).

Other analytes include, but are not limited to, herbicides such as triazines, haloacetanilides, carbamates, toluidines, and diphenyl ethers, including available herbicides such as phenoxyl alkanoic acids, bipyridiniums, benzonitriles, dinitroanilines, acid amides, carbamates, thiocarbamates, heterocyclic nitrogen compounds including triazines, pyridines, pyridazinones, sulfonylureas, imidazoles, substituted areas, halogenated aliphatic carboxylic acids, inorganics, organometallics and derivatives of biologically important amino acids; agents of war that are members of the group consisting of mustard and related vesicants including the agents known as HD, Q, T, HN1, HN2, HN3, isopropyl methylphosphonofluoridate, soman pinacolyl, and methylphosphonofluoridate; incapacitants such as BZ, 3-quinuclidinyl benzilate and irritants such as the riot control compound CS, bactericides (e.g., formaldehyde), fumigants (e.g., bromomethane), fungicides (e.g., 2phenylphenol, biphenyl, mercuric oxide, imazalil), acaricides (e.g., abamectin, bifenthrin); noxious gases such as CO, CO₂, SO₃, H₂SO₄, SO₂, NO, NO₂, N₂O₄ and the like.

In still further embodiments, analytes detectable by the systems disclosed herein include volatile organic compounds such as pentane, hexane, heptane, 2-butenal, ethanol, 3-methyl butanal, 4-methyl pentanone, hexanal, heptanal, 2-pentyl furan, and octanal and VOCs, gasoline components and additives, industrial chemicals, solvents, and degreasing agents such as Benzene, Dichloromethane (Methylene chloride); trichloroethylene, tetrachloroethylene (perchloroethylene), methyl tertiary butyl ether, chloroform, bromodichloromethane, chlorodibromomethane, bromoform, onochloroacetic acid, dichloroacetic acid, trichloroacetic acid, monobromoacetic acid, dibromoacetic acid, Carbon tetrachloride, Chlorobenzene, 1,2-Dichlorobenzene, 1,4-Dichlorobenzene, 1,2-Dichloroethane, cis-Dichloroethylene, trans-Dichloroethylene, Dichloromethane, 1,2-Dichloropropane, Ethylbenzene, Styrene, Tetrachloroethylene, 1,1,1-Trichloroethane, Trichloroethylene, Toluene, 1,2,4-Trichlorobenzene, 1,1-Dichloroethylene, 1,1,2-Trichloroethane, Vinyl chloride, Xylenes, 2,3,7,8-TCDD (dioxin), 2,4-D, 2,4,5-TPAlachlor, Heptachlor, Heptachlor Epoxide, Hexachlorobenzene, Hexachlorocyclopentadiene, Lindane, Methoxychlor, Oxamyl, PCBs (as decachlorobiphenyl), PCBs (as Aroclors), Pentachlorophenol, Picloram, Simazine, and Toxaphene.

Other analytes include, but are not limited to, explosive agents including, but not limited to, Acetone Peroxide, Allyl Hydroperoxide, Ammonium Nitrate, Ammonium Picrate, Astrolite, Benzalaminoguanidine Nitrate, a-Benzenediazobenzyl Hydroperoxide, DADNBU, DADNPE, DDNP, Dinitrobenzene, Dinitrochlorobenzene, Dinitropolystyrene, DNPA, EGDN (ethylene glycol dinatrate), FOX-7, Guanidine Carbonate, Guanidine Nitrate, 1,1,1,3,5,5,5-Heptanitropentane, Hexamethylenetetramine Dinitrate, Hexanitrocarbanilide, Hexanitrodiphenylamine, HMTD, HMX (Octogen), HNIW, HNO, IPN, Lead Azide, Lead Nitratophosphite, Lead Picrate, Lead Styphnate, Lead 2,4,6-Trinitro-3, Oxybenzoate, Maltose Octanitrate, Mannitol Hexanitrate, MEDINA, MEDNA, MeEDNA, Mercurous Nitratophosphite, Mercury Fulminate, 2-Methyl-2-Nitro-1 PropanoInitrate, Metriol Trinitrate, MMAN, NIBGkDN, NIBGTN, Nitrated Petroleum, m-Nitrobenzenediazonium Perchlorate, 2-Nitro-2-(3,5-dinitrophenyl)-propanediol-1,3 Dinitrate, Nitrogen Sulfide, Nitrogen Trichloride, Nitrogen Triiodide, Nitroglycerin, Nitroguanidine, 2-Nitro-2-(m-Nitrophenyl)-Propanediol-1,3 Dinitrate, Nitrosoguanidine, Nitrostarch,Nitrosyl Perchlorate, NONA, NPN, NTN, Perchlorates, N-Perchlorylpiperidine, PETN (Pentaerythritol tetranitrate, including Detasheet), Petrin, Petrin Acrylate, PGDN, 1-Phenyl-2-Nitro-1-Propene, Picric Acid, m-Picrylpicryl Chloride, Potassium Picrate, Propylpicrate, PVN, RDX (Hexogen, including C-4), Semtex (RDX plus PETN), Silver Fulminate, TACC, TATB (1,3,5-triamino-2,4,6-trinitrobenzene), TeNN, Tetracene, Tetranitromethane, Tetryl, TNO, TNPEN, TNPht, Triacetone triperoxide, Trinitroanisol, Trinitrobenzene, Trinitro-m-Cresol, Trinitromethane, 2,4,6-Trinitro-m-Phenylenediamine, 1,1,1-Trinitro-2-Propyl Acrylate, Trinitrostilbene, Trinitrotoluene, Tris[1,2-Bis (Difluoramino)-Ethyl]Isocyanurate, and Urea Nitrate, and tags for explosive compounds including, but not limited to, EGDN (ethylene glycol dinitrate), DMNB (2,3-dimethyl-2,3-dinitrobutane), o-MNT, p-MNT, 2,4-dinitrotoluene, 1,3-dinitrobenzene, 3,5-dinitrotoluene, 1,4-dinitrobenzene, 1,2-dintrobenzene, ethylbenzene, m-Xylene, styrene, nonane, alpha-pinene, decane, ethyl hexanol, undecane, dodecane, tridecane, cyclohexanone, toluene, and 2-ethyl-1-hexanol.

Other detectable analytes include, but are not limited to, Acrolein, Benzyl Bromide, Benzyl Chloride, Benzyl Iodide, Bromoacetone, Bromoacetophenone, Bromobenzyl Cyanide, Bromopicrin, Carbon Monoxide, Carbonyl Bromide, Chloroacetone, Chloroacetophenone, Chloroformoxime, Chloromethyl, Chloroformate, Chloropicrin, Chlorosulfonic Acid, Cyanogen Bromide, Cyanogen Chloride, Cyanogen Fluoride, Cyanogen Iodide, Cyclosarin, Cyclosoman, Dibromoacetylene, Dibromomethyl Ether, Dibromoethyl Sulfide, Dichloroethyl Sulfide, Dichloroformoxime, Dichloromethyl Chloroformate, Dichloromethyl Ether, Diiodoacetylene, Diiodoethyl Sulfide, Dimethyl Sulfate, Diphenyl Chloroarsine, Diphenyl Cyanoarsine, Ethyl Bromoacetate, Ethyl Chloroacetate, Ethyl Dihloroarsine, Ethyl Iodoacetate, Hexachloromethyl Carbonate, Hydrocyanic Acid, Lewisite, Methyl Chloroformate, Methyl Chlorosulfonate, Methyl Dihloroarsine, Methyl Fluorosulfonate, Methyl Formate, Methyl Sulfuric Acid, Oxalyl Chloride, Perchloromethyl Mercaptan, Phenarsazine Chloride, Phenyl Carbylamine Chloride, Phenyl Dichloroarsine, Phosgene, Sulfuryl Chloride, Tetrachlorodinitroethane, Thiophosgene, Thiosarin, Thiosoman, Trichloromethyl Chloroformate, Trichloro Nitroso Methane, and Xylyl Bromide.

It is contemplated that the devices of the present invention utilize a number of different recognition moieties for detecting the analytes described above. In some embodiments, the devices comprise a single type of recognition moiety, while in other embodiments, the devices comprise two or different types of recognition moieties to provide a multiplex assay device.

In some embodiments of the present invention, a “recognition moiety” attached to or associated with the substrate is utilized to bind to or otherwise interact with another molecule or molecules (e.g., analytes). For example, in some embodiments, recognition moieties are attached to either ω-functionalized spacer arms or co-functionalized SAM components which are in turn attached to or associated with the substrate. Furthermore, a recognition moiety can be presented by a polymer surface (e.g., a rubbed polymer surface).

In some preferred embodiments, the recognition moiety comprises an organic functional group. In presently preferred embodiments, the organic functional group is a member selected from the group consisting of amines, carboxylic acids, drugs, chelating agents, crown ethers, cyclodextrins or a combination thereof.

In other preferred embodiments, the recognition moiety is a metal ion as described above. Preferred metal ions include Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺ and combinations thereof.

In another preferred embodiment, the recognition moiety is a biomolecule. In still further preferred embodiments, the biomolecule is a protein, antigen binding protein, peptide, nucleic acid (e.g., single nucleotides or nucleosides, oligonucleotides, polynucleotides and single- and higher-stranded nucleic acids) or a combination thereof. In a presently preferred embodiment, the recognition moiety is biotin. In some embodiments of the present invention, the recognition moiety is an antigen binding protein. Such antigen binding proteins include, but are not limited to polyclonal, monoclonal, chimeric, single chain, Fab fragments, and Fab expression libraries.

Various procedures known in the art may be used for the production of polyclonal antibodies. For the production of antibody, various host animals, including but not limited to rabbits, mice, rats, sheep, goats, etc., can be immunized by injection with the peptide corresponding to an epitope. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature 256:495-497 [1975]), as well as the trioma technique, the human B-cell hybridoma technique (See e.g., Kozbor et al., Immunol. Tod., 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In addition, it is contemplated that techniques described for the production of single chain antibodies (U.S. Pat. No. 4,946,778; herein incorporated by reference) will find use in producing specific single chain antibodies that serve as recognition moieties. Furthermore, it is contemplated that any technique suitable for producing antibody fragments will find use in generating antibody fragments that are useful recognition moieties. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent. In still further embodiments, the recognition moiety comprises a phage displaying an antigen binding protein.

In some embodiments where the recognition moiety is a polynucleotide or polypeptide, a plurality of recognition moieties are arrayed on the substrates using photo activated chemistry, microcontact printing, and ink-jet printing. In particularly preferred embodiments, photolithography is utilized (See e.g., U.S. Pat. Nos. 6,045,996; 5,925,525; and 5,858,659; each of which is herein incorporated by reference). Using a series of photolithographic masks to define substrate exposure sites, followed by specific chemical synthesis steps, the process constructs high-density arrays of oligonucleotides, with each probe in a predefined position in the array. Multiple probe arrays are synthesized simultaneously on, for example, a large glass wafer. The wafers are then diced, and individual probe arrays are packaged in injection-molded plastic cartridges, which protect them from the environment and serve as chambers for hybridization.

In other embodiments, nucleic acid recognition moieties are electronically captured on a suitable substrate (See e.g., U.S. Pat. Nos. 6,017,696; 6,068,818; and 6,051,380; each of which are herein incorporated by reference). Through the use of microelectronics, this technology enables the active movement and concentration of charged molecules to and from designated test sites on its semiconductor microchip. DNA capture probes unique to a given target are electronically placed at, or “addressed” to, specific sites on the microchip. Since DNA has a strong negative charge, it can be electronically moved to an area of positive charge.

In still further embodiments, recognition moieties are arrayed on a suitable substrate by utilizing differences in surface tension (See e.g., U.S. Pat. Nos. 6,001,311; 5,985,551; and 5,474,796; each of which is herein incorporated by reference). This technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

In still further embodiments, recognition moieties are spotted onto a suitable substrate. Such spotting can be done by hand with a capillary tube or micropipette, or by an automated spotting apparatus such as those available from Affymetrix and Gilson (See e.g., U.S. Pat. Nos. 5,601,980; 6,242,266; 6,040,193; and 5,700,637; each of which is incorporated herein by reference).

When the recognition moiety is an amine, in preferred embodiments, the recognition moiety will interact with a structure on the analyte which reacts by binding to the amine (e.g., carbonyl groups, alkylhalo groups). In another preferred embodiment, the amine is protonated by an acidic moiety on the analyte of interest (e.g., carboxylic acid, sulfonic acid).

In certain preferred embodiments, when the recognition moiety is a carboxylic acid, the recognition moiety will interact with the analyte by complexation (e.g., metal ions). In still other preferred embodiments, the carboxylic acid will protonate a basic group on the analyte (e.g. amine).

In another preferred embodiment, the recognition moiety is a drug moiety. The drug moieties can be agents already accepted for clinical use or they can be drugs whose use is experimental, or whose activity or mechanism of action is under investigation. The drug moieties can have a proven action in a given disease state or can be only hypothesized to show desirable action in a given disease state. In a preferred embodiment, the drug moieties are compounds which are being screened for their ability to interact with an analyte of choice. As such, drug moieties which are useful in practicing the instant invention include drugs from a broad range of drug classes having a variety of pharmacological activities.

Classes of useful agents include, for example, non-steroidal anti-inflammatory drugs (NSAIDS). The MAIDS can, for example, be selected from the following categories: (e.g., propionic acid derivatives, acetic acid derivatives, fenamic acid derivatives, biphenylcarboxylic acid derivatives and oxicams); steroidal anti-inflammatory drugs including hydrocortisone and the like; antihistaminic drugs (e.g., chlorpheniranune, triprolidine); antitussive drugs (e.g., dextromethorphan, codeine, carmiphen and carbetapentane); antipruritic drugs (e.g., methidilizine and trimeprizine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyraminde, quinidine, encainide); P-adrenergic blocker drugs (e.g., metoprolol, acebutolol, betaxolol, labetalol and timolol); cardiotonic drugs (e.g., milrinone, amrinone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltazem, amiodarone, isosuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chlorprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazapam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole; pentamidine, gentamycin, kanamycin, lineomycin, methacycline, methenamine, minocycline, neomycin, netilmycin, paromomycin, streptomycin, tobramycin, miconazole, and amanfadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, a-2-interferon) anti-estrogens (e.g., tamoxifen), antimetabolites (e.g., fluorouracil, methotrexate, mercaptopurine, thioguanine).

The recognition moiety can also comprise hormones (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, dephenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progenstogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful recognition moieties include immunomodulating drugs (e.g., antihistamines, mast cell stabilizers, such as Iodoxamide and/or cromolyn, steroids (e.g., triamcinolone, beclomethazone, cortisone, dexamethasone, prednisolone, methylprednisolone, beclomethasone, or clobetasol), histamine H₂ antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

When the recognition moiety is a chelating agent, crown ether or cyclodextrin, host-guest chemistry will dominate the interaction between the recognition moiety and the analyte. The use of host-guest chemistry allows a great degree of recognition-moiety-analyte specificity to be engineered into a device of the invention. The use of these compounds to bind to specific compounds is well known to those of skill in the art. See, for example, Pitt et al. “The Design of Chelating Agents for the Treatment of Iron Overload,” In, INORGANIC CHEMISTRY IN BIOLOGY AND MEDICINE; Martell, A. E., Ed.; American Chemical Society, Washington, D.C., 1980, pp. 279-312; Lindoy, L. F., THE CHEMISTRY OF MACROCYCLIC LIGAND COMPLEXES; Cambridge University Press, Cambridge, 1989; Dugas, H., BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, R. E., Whitaker, 1.R., Eds., American Chemical Society, Washington, D.C., 1982, pp. 370-387; Kasina et al. Bioconjugate Chem. 9:108-117 (1998); Song et al., Bioconjugate Chem. 8:249-255 (1997).

In a presently preferred embodiment, the recognition moiety is a polyaminocarboxylate chelating agent such as ethylenediaminetetraacetic acid (EDTA) or diethylenetriaminepentaacetic acid (DTPA). These recognition moieties can be attached to any amine-terminated component of a SAM or a spacer arm, for example, by utilizing the commercially available dianhydride (Aldrich Chemical Co., Milwaukee, Wis.).

In still further preferred embodiments, the recognition moiety is a biomolecule such as a protein, nucleic acid, peptide or an antibody. Biomolecules useful in practicing the present invention can be derived from any source. The biomolecules can be isolated from natural sources or can be produced by synthetic methods. Proteins can be natural proteins or mutated proteins. Mutations can be effected by chemical mutagenesis, site-directed mutagenesis or other means of inducing mutations known to those of skill in the art. Proteins useful in practicing the instant invention include, for example, enzymes, antigens, antibodies and receptors. Antibodies can be either polyclonal or monoclonal. Peptides and nucleic acids can be isolated from natural sources or can be wholly or partially synthetic in origin.

In those embodiments wherein the recognition moiety is a protein or antibody, the protein can be tethered to a SAM component or a spacer arm by any reactive peptide residue available on the surface of the protein. In preferred embodiments, the reactive groups are amines or carboxylates. In particularly preferred embodiments, the reactive groups are the e-amine groups of lysine residues. Furthermore, these molecules can be adsorbed onto the surface of the substrate or SAM by non-specific interactions (e.g., chemisorption, physisorption).

Recognition moieties that are antibodies can be used to recognize analytes that are proteins, peptides, nucleic acids, saccharides or small molecules such as drugs, herbicides, pesticides, industrial chemicals and agents of war. Methods of raising antibodies for specific molecules are well-known to those of skill in the art. See, U.S. Pat. Nos. 5,147,786; 5,334,528; 5,686,237; 5,573,922; each of which is incorporated herein by reference. Methods for attaching antibodies to surfaces are also art-known (See, Delamarche et al. Langmuir 12:1944-1946 (1996)).

Peptides and nucleic acids can be attached to a SAM component or spacer arm. Both naturally-derived and synthetic peptides and nucleic acids are of use in conjunction with the present invention. These molecules can be attached to a SAM component or spacer arm by any available reactive group. For example, peptides can be attached through an amine, carboxyl, sulfhydryl, or hydroxyl group. Such a group can reside at a peptide terminus or at a site internal to the peptide chain. Nucleic acids can be attached through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain (See, Chrisey et al. Nucleic Acids Res. 24:3031-3039 (1996)).

When the peptide or nucleic acid is a fully or partially synthetic molecule, a reactive group or masked reactive group can be incorporated during the process of the synthesis. Many derivatized monomers appropriate for reactive group incorporation in both peptides and nucleic acids are know to those of skill in the art (See, for example, THE PEPTIDES: ANALYSIS, SYNTHESIS, BIOLOGY, Vol. 2: “Special Methods in Peptide Synthesis,” Gross, E. and Melenhofer, J., Eds., Academic Press, New York (1980)). Many useful monomers are commercially available (Bachem, Sigma, etc.). This masked group can then be unmasked following the synthesis, at which time it becomes available for reaction with a SAM component or a spacer arm.

In other preferred embodiments, the peptide is attached directly to the substrate (See, Frey et al. Anal. Chem. 68:3187-3193 (1996)). In a particularly preferred embodiment, the peptide is attached to a gold substrate through a sulfhydryl group on a cysteine residue. In another preferred embodiment, the peptide is attached through a thiol to a spacer arm that terminates in, for example, an iodoacetamide, chloroacetamide, benzyl iodide, benzyl bromide, alkyl iodide or alkyl bromide. Similar immobilization techniques are known to those of skill in the art (See, for example, Zull et al. J. Ind Microbiol. 13:137-143 (1994)).

In another preferred embodiment, the recognition moiety forms an inclusion complex with the analyte of interest. In a particularly preferred embodiment, the recognition moiety is a cyclodextrin or modified cyclodextrin. Cyclodextrins are a group of cyclic oligosaccharides produced by numerous microorganisms. Cyclodextrins have a ring structure which has a basket-like shape. This shape allows cyclodextrins to include many kinds of molecules into their internal cavity (See, for example, Szejtli, J., CYCLODEXTRINS AND THEIR INCLUSION COMPLEXES; Akademiai Klado, Budapest, 1982; and Bender et al., CYCLODEXTRIN CHEMISTRY, Springer-Verlag, Berlin, 1978).

Cyclodextrins are able to form inclusion complexes with an array of organic molecules including, for example, drugs, pesticides, herbicides and agents of war (See, Tenjarla et al., J. Pharm. Sci. 87:425-429 (1998); Zughul et al., Pharm. Dev. Technol. 3:43-53 (1998); and Albers et al., Crit. Rev. Ther. Drug Carrier Syst. 12:311-337 (1995)). Importantly, cyclodextrins are able to discriminate between enantiomers of compounds in their inclusion complexes. Thus, in one preferred embodiment, the invention provides for the detection of a particular enantiomer in a mixture of enantiomers (See, Koppenhoefer et al. J. Chromatogr. A 793:153-164 (1998)).

The cyclodextrin recognition moiety can be attached to a SAM component, through a spacer arm or directly to the substrate (See, Yamamoto et al., J. Phys. Chem. B 101:6855-6860 (1997)). Methods to attach cyclodextrins to other molecules are well known to those of skill in the chromatographic and pharmaceutical arts (See, Sreenivasan, Appl. Polym. Sci. 60:2245-2249 (1996)).

A. Reflection Based Probing

In some embodiments, the devices of the present invention form a Fabry-Perot filter. The present invention is not limited to a particular mechanism. Indeed, an understanding of the mechanism of the present invention is not needed to practice the invention. Nevertheless, when electromagnetic radiation propagates through an interface between two dielectric media it undergoes reflection at the interface. If a dielectric material is sandwiched between two highly reflecting mirrors forming a cavity, multiple reflection of radiation occurs in the cavity. For a given thickness and the dielectric properties of the cavity, the reflected electromagnetic radiation interferes constructively and shows a maximum at a particular wavelength. The wavelength at which the reflected radiation shows peak intensity depends on the thickness of cavity and the dielectric property of the cavity. When the refractive index of the cavity changes, the wavelength at which the maximum reflection occurs also changes. If the mirrors are functionalized with receptors targeted to the specific analyte, binding of the target induces an orientational transition of the LC and hence a change in the dielectric property of the cavity. This change in the dielectric constant results in a shift in the wavelength at which the reflected intensity is maximum.

In some embodiments, the Fabry-Perot filter devices comprise a first surface (e.g., an interior surface) that displays at least one recognition moiety. In some embodiments, the surface is reflective. In preferred embodiments, the first surface is gold. In some preferred embodiments, the gold is deposited on a supporting substrate, such as glass or silicon. Other suitable substrates are described in more detail above. In some embodiments, the gold surface is functionalized with an organic layer with which the recognition moiety interacts. Preferred organic layers include, but are not limited to, aminothiophenol (ATP), 4-mercaptobenzoic acid (MBA), 2-mercaptoethylamine-HCl (MEA), and 11-mercaptoundecanoic acid (MUA). In further preferred embodiments, the devices comprise a second surface coated in a reflective material, preferably gold. In some embodiments, the second surface also displays a recognition moiety. In some embodiments, the first and second surfaces are configured opposite one another to form a chamber there between. Preferably, the chamber is fillable with a liquid crystal. Preferred mesogens forming the liquid crystal are listed above and include, but are not limited to, E7 (4-pentyl-4′-cyanobiphenyl), MLC, 5CB (4-n-pentyl-4′-cyanobiphenyl), and 8CB (4-cyano-4′octylbiphenyl).

In still further embodiments, at least one recognition moiety is attached or otherwise interacts with the organic layer. The present invention is not limited to any particular recognition moiety. Indeed, a variety of recognition moieties may be utilized, including, but not limited to, an organic functional group selected from the group consisting of amines, carboxylic acids, drugs, chelating agents, crown ethers, cyclodextrins or a combination thereof, a biomolecule selected from the group consisting of a protein, antigen binding protein such as a monoclonal antibody, polyclonal antibody, chimeric antibody, humanized antibody, Fab fragment, single chain antibody, etc., peptide, nucleic acid (e.g., single nucleotides or nucleosides, oligonucleotides, polynucleotides and single- and higher-stranded nucleic acids) or combinations thereof, and metal ions selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺ and combinations thereof.

The present invention is not limited to any particular substrate shape. Indeed, a variety of substrate shapes are contemplated, including, but not limited to, discs, cylinders, and spheres. Disc shaped devices are preferably configured as described above, with two planar surfaces opposed to one another to create a chamber that is fillable with a liquid crystal. In preferred embodiments, the discs are small enough so that they tumble when released into the atmosphere. For example, in some embodiments, the discs have a diameter of between about 0.1 mm to 10 cm, preferably about 1 mm to about 100 mm. In some preferred embodiments, highly reflecting mirrors are prepared by depositing 500 nanometer thick gold films on clean glass slides (or plastic films) using electron beam evaporator. In further preferred embodiments, these gold mirrors are functionalized with an organic layer such as 4-aminothiophenol (ATP), 4-mercaptobenzoic acid (MBA), 2-mercaptoethylamine-HCl (MEA), or 11-mercaptoundecanoic acid (MUA) all at 1 mM concentration in ethanol. In still further preferred embodiments, the organic layer is then treated with receptors specific to the target analyte (for example, gallium perchlorate or indium perchlorate for Diazianon and iron perchlorate and lead perchlorate for Malathion). In further embodiments, glass fiber rods with approximately a 25 micron diameter mixed in isopropanol are sprayed uniformly over one of the functionalized mirrors. These rods act as spaces defining the thickness of the dielectric cavity. An optical cell is fabricated forming a cavity between two reflecting mirrors. In some embodiments, the mirrors are glued together using UV curable adhesives. The cavity is then filled with the liquid crystal, such as 4-n-pentyl-4′-cyanobiphenyl (5CB). The present invention is not limited to a particular mechanism of action. Indeed an understanding of the mechanism of action is not necessary to practice the present invention. Nevertheless, without exposure to the target analyte, the liquid crystal aligns in one preferred direction determined by the receptors on the mirrors (for example perpendicular to the surface for the treatments described above). In some embodiments, the mirror assembly is placed in the path of the light in a spectrometer. For the optimized thickness, a peak appears in the transmitted intensity at a particular wavelength determined by the ordinary refractive index of the LC materials. In some embodiments, upon exposure to an analyte (such as Malathion, or Diazinon), the liquid crystal undergoes an orientational transition to the random planar orientation, where liquid crystal molecules are oriented parallel to the surface in random azimuthal direction. In some preferred embodiments, the device is placed in a light path. A peak appears at a wavelength that corresponds to the average refractive of the random planar orientation. The shift in the peak position of the transmission spectrum indicates a change in the refractive index of the cavity caused by the orientational transition of the liquid crystal that is induced by binding of the analyte to the receptor on the surface.

In other preferred embodiments, hollow polymer cylinders (about 100 to 1000 microns in diameter, and preferably about 500 micron in diameter; about 1 mm to 1 cm in length, preferably about 5 mm in length) are first coated with a reflective material such as gold. Preferably the coating is from about about 50 to about 1000 nm in thickness, and most preferably about 500 nm thick. A spacer is then formed on the cylinder. In some embodiments, the spacer is from about 50 to about 200 microns in thickness, preferably about 25 microns. In some preferred embodiments, the spacer comprises glass fiber rods with desired diameter preferably 25 microns (such as from EM Industries) or plastic micropearls (spheres) of desired diameter preferably 25 micron (such as from Sekesui Chemicals, Hong Kong). In some preferred embodiments, these spacers are mixed in isopropyl alcohol and then sprayed on to the cylinders. A ˜25 micron sacrificial layer of photoresist is then coated to these cylinders. Examples of useful photoresist layers include, but are not limited to SU8 2010 from Michochem. Another thin nanoporous layer of gold is deposited on top of the sacrificial layer. The gold film with nanopores is strongly reflecting but allows small molecules to penetrate through it. The sacrificial layer is then dissolved in acetone. The spacers in between two gold surfaces act as supporting struts. These hollow cylinders are then functionalized with an organic layer and the receptor chemistry as described above and filled with a liquid crystal. In still further embodiments, the first surface is spherical and a second surface and chamber are formed as described for the cylinder embodiments.

In other embodiments, the devices of the present invention form a rugate filter. Again, the present invention is not limited to a particular mechanism. Indeed, an understanding of the mechanism of the present invention is not needed to practice the invention. Nevertheless, as the electromagnetic radiation propagates through a number of interfaces between dielectric layers, multiple reflections occur at each interface and a portion of the radiation is transmitted and a portion of it is reflected. If the dielectric constant of the medium exhibits sinusoidal variation, then the reflected intensity shows a peak in the reflected intensity at a wavelength that depends on the average dielectric constant and the amplitude of sinusoidal variation of dielectric constant. The position of the reflected peak in the electromagnetic spectrum shifts as the average refractive index of the sinusoidal variation changes. Accordingly, in some embodiments, a sinusoidal variation in the dielectric property is created by fabricating porous silicon with sinusoidal porosity gradient along the depth. See, e.g., Li et al., Science 299:2045-47 (2003); Seals et al., J. Applied. Phys. 91(4):2519-23 (2002); Schmedake et al., Adv. Mater. 14(18):1270-72 (2002); Link and Sailor, Proc. Nat'l. Acad. Sci USA 100(19):10607-10 (2003), all of which are incorporated herein by reference in their entirety. When the pores are functionalized with the receptors specific to the target analyte and then filled with LCs, they align LC in a specific orientation. Upon exposure to the target analyte the LC undergoes an orientational transition, which induces a change in the dielectric constant of the pores resulting in a shift in the position of the peak.

The present invention is not limited to the use of any particular type of silicon substrate. In some embodiments, the silicon substrate is p-type, boron doped silicon wafer with about a 1 mOhm cm resistivity and polished on 100 face. The silicon substrate is preferably ultrasonicated in isopropanol and then rinsed with water. In some embodiments, the silicon wafers are etched using an anodization-etching process with a mixture of 48% hydrofluoric acid and absolute ethanol (1:3 by volume) in a Teflon cell using a sinusoidally modulated current density to generate a sinusoidal variation in the porosity gradient. In further embodiments, the amplitude, period, and duration of the sinusoidal current density is adjusted to achieve the optimum porous size and distribution. It will be recognized that these parameters can be varied and optimized for the detection of different analytes. In still further embodiments, the current density is then ramped up so that a freestanding film of the porous silicon is detached from the substrate.

In some embodiments, the pores created in the silicon substrate are functionalized with an organic layer, preferably with silane containing moieties as described above. In preferred embodiments, the porous film of silicon is mounted on a glass slide and in a solution of 3-aminopropyltriethoxy silane (APES) in acetone (˜2% by volume) to functionalize inner surface of the pores. In preferred embodiments, a suitable recognition moiety (for example, one of the recognition moieties described above) is reacted with or added to the organic layer. In some preferred embodiments, the liquid crystal aligns perpendicular to the surface of the pores when added. In further embodiments, the porous silicon is mounted on the spectrophotometer to measure the reflectivity spectrum covering both infrared and visible range. Depending on the dimension of the pores the reflected spectrum exhibits a peak at a particular wavelength that depends on the pore size distribution and the dielectric constant of the material in the pores. Since the alignment of the liquid crystal on the pores is perpendicular to the pore's surface, the molecular distribution will be radial. In further embodiments, the porous silicon is exposed to the target analyte. The present invention is not limited to any particular mechanism of action. Indeed, an understanding of the mechanism of action is not necessary to practice the present invention. Nevertheless, as the target analyte binds to the capturing agent on the surface of the pores, the liquid crystal undergoes an orientational transition to planar alignment (liquid crystal molecules align parallel to the surface). This causes a change in the dielectric property of the pores. In some embodiments, the porous silicon is again analyzed for the reflectivity spectrum. A shift in the peak position of the reflectivity profile indicates the binding of the target analyte to the receptor.

In still further embodiments, devices such as those described above are irradiated with electromagnetic radiation from the radio frequency region, including, but not limited to, frequencies between 1 KHz and 10 THz, and including the VLF, LF, MF, HF, VHF, UHF, SHF and EHF regions of the radio spectrum. Studies have demonstrated that analysis of the reflection and/or transmission spectra of RF radiation can be used to identify analytes. See, e.g., U.S. Pat. Appl. 2004086929, Choi et al., Int'l. J. High Speed Electronics and Systems 13(4):937-950 (2003); van der Weide, Springer Series in Optical Sciences (2003), 85:317-334 (2003), all of which are incorporated herein by reference. In some preferred embodiments of the invention, a change in orientation of a liquid crystal gives rise to a change in the reflection or transmission spectra of RF radiation. In further preferred embodiments of the invention, the frequency of the radiation is in the 0.1-10 THz range. Methods known to those skilled in the art are used to analyze the radiation returned to a detector following interaction with the liquid crystal.

B. Photoluminescence

In some embodiments, a change in the orientation of a liquid crystal is detected by photoluminescence. The present invention is not limited to a particular mechanism of action. Indeed, an understanding of the mechanism of action is not necessary to understand the present invention. Nevertheless, when silicon with nanometer scale porous structure is exposed to electromagnetic radiation at short wavelength, typically in the ultraviolet region, electron-hole pairs are created. These excess carriers subsequently recombine radiating electromagnetic radiation. As the characteristic size of the structures in the porous silicon decreases to the nanometer scales, the band gap of the silicon nanostructures progressively widens. The recombination of these quantum confined carriers (electron-hole pair) in the wide band gap causes emission of electromagnetic radiation in the visible region. The wavelength of the emitted light depends on the dielectric constant of the materials filling the pores, besides the detailed structure of the pores themselves. When the surfaces of the pores are functionalized with the receptors specific to the target analyte the liquid crystal aligns perpendicular to the surface of the pores. The porous silicon then emits light at a wavelength that corresponds to the radial distribution of the liquid crystal molecules. When the target analyte binds to the receptors on the surface of the pores, liquid crystal aligns parallel to the surface of the pores causing a change in the dielectric constant. This results in a change in the position of the peak. It will be recognized that the present invention is not limited to any particular type of change in liquid crystal orientation and that the described change from perpendicular to parallel is exemplary. Other changes are also contemplated, including, for example, from parallel to perpendicular or changes in the amount of twist, where, for example, chiral nematic liquid crystals are utilized.

In some embodiments, porous silicon substrates are fabricated and functionalized as described above. In further embodiments, the porous silicon is illuminated by a UV light. The exact wavelength of the UV light depends on the actual porous size, porous size distribution and the refractive index of the liquid crystal material. The photoluminescence of the porous silicon is measured using UV-Visible spectrophotometer. The spectrum shows a peak at a wavelength corresponding to the orientation of the liquid crystal. In some preferred embodiments, the porous silicon is now exposed to the target analyte in a closed chamber. The present invention is not limited to any particular mechanism of action. Indeed, an understanding of the mechanism of action is not necessary to understand the present invention. Nevertheless, as the target analyte binds to the capturing agent on the surface of the pores the LC undergoes an orientational transition. It is contemplated that the change in the orientation of thje liquid crystal corresponds to a change in the spectrum of radiation emitted by the porous silicon.

C. Fluorescence Based Detection

In other embodiments, stand-off detection is accomplished using a fluorescent reporter system. The present invention is not limited to any particular mechanism of action. Indeed, an understanding of the mechanism of action is not necessary to understand the present invention. Nevertheless, certain compounds such as 4-(4-dihexadecylsminostyryl)-N-methylpyridinium iodide (DIA), 1,3,5,7,8-pentamethyl-2,6,-di-t-butylpyrromethane-difluoreborate PM-597, 4-(dicynaomethylene)-2-methyl-6-(4-dimethylamino styryl)-4H-pyran (DCM), eurobium(III) thenoyltrifluoroacetonate trihydrate [Eu(TTA)₃.H₂O] etc., when dissolved in liquid crystal emit visible light upon exposure to UV light. The intensity and the wavelength of the emitted light depend on the orientation of the dye molecules with respect to liquid crystal orientation. If the dye molecules are immobilized on to the surface in a fixed orientation with respect to the surface and the liquid crystal undergoes orientational change, the characteristics of the emitted radiation changes. When analyte binds to the receptor, the liquid crystal undergoes orientational transition, the wavelength of the emitted light changes.

Accordingly, in some embodiments, a thin gold film is deposited on a substrate (preferably a UV transparent quartz substrate or plastic film) using an electron beam evaporator. The gold surface are functionalized and treated with a recognition moiety as described above and assembled into a liquid crystal assay device using small glass spacer rods as described above. The device is then filled with a liquid crystal. The present invention is not limited to any particular mechanism of action. Indeed, an understanding of the mechanism of action is not necessary to understand the present invention. Nevertheless, without exposure to the target analyte, the liquid crystal aligns in one preferred direction determined by receptors on the surfaces, for example, perpendicular to the surface for the types of treatments described above. In preferred embodiments, the optical cell is irradiated with UV light, which in some preferred embodiments is provided by a laser. In the absence of the analyte the fluorescent molecules emit visible light at a wavelength that corresponds to the homeotropic alignment of liquid crystals. When the device is exposed to an analyte (such as Malathion, or Diazinon) the liquid crystal undergoes orientational transition to, for example, a random planar configuration where liquid crystal molecules are oriented parallel to the surface in random azimuthal direction. The shift in the peak position of the fluorescence spectrum (change in the color of the emitted light) indicates a change in the dielectric environment of the fluorescent molecules on the surface. This change is caused by the orientational transition of the liquid induced by binding of the analyte to the receptor on the surface.

In other embodiments of the invention, fluorescent dye molecules such as Acridine Orange Base, Rhodamine 6G, perchlorate,5-decyl-4,4-difluoro-4-bora-3a,4a-diaza-s-indacene-3-propionic acid, Nile Red, N,N′ Bis(2,5-di-tert-butylphenyl)-3,4,9,10-perylenedicarboximide, etc., are dissolved into the liquid crystal forming a guest-host system. The present invention is not limited to any particular mechanism of action. Indeed, an understanding of the mechanism of action is not necessary to understand the present invention. Nevertheless, the orientation of the dye molecule, in general, is parallel to the alignment of the LC molecules in the LC cell. When a beam of light (typically in the visible region) with its polarization parallel to the transition dipole moment of the dye, is passed through the guest host system, it gets absorbed by the dye molecule. The dye molecules then radiate visible light at a different wavelength. However, if the incident light has its polarization perpendicular to the transition dipole moment of the dye molecule it is not absorbed and the dye molecule do not emit any radiation. Therefore, in the absence of the target analyte, when the guest-host system is interrogated by a polarized light corresponding to the excitation wavelength of the dye used, the light coming from the system is composed of the excitation wavelength. If the analyte is present in the ambient, it interacts with the functionalized surface and the liquid crystal undergoes orientational transition from the homeotropic to the random planar orientation. This causes a rotation of the transition moment of the dye molecule parallel to the polarization direction of the excitation beam. The dye molecules then absorb the incident wavelength and emit light at different wavelength. Thus, by probing liquid crystal-dye mixture using polarized light propagating perpendicular to the cell surface, the presence of the analyte in the environment can be probed. The liquid crystal assay device cell is fabricated as described above except that it is filled with liquid crystal-dye mixture. For the interrogation, the polarization can be integrated on the liquid crystal cell or can be probed by sending polarized light.

In still further embodiments, the fluorescent properties of quantum dots are utilized for standoff detection of analytes. The present invention is not limited to any particular mechanism of action. Indeed, an understanding of the mechanism of action is not necessary to understand the present invention. Nevertheless, some semiconductor quantum dots with nanometer size emit visible light when exposed to UV radiation. Due to the quantum confinement the electron-hole pairs trapped at the surface have a large a large band gap. Because of this large band gap, these semiconductor quantum dots absorb light in the UV region. The wavelength of light emitted by these fluorescence particles depends, besides their size, on properties of the surrounding medium, such as but not limited to, the dielectric constant of the surrounding medium. In some preferred embodiments, these quantum dots are functionalized with the receptors targeted for the analyte so that a liquid crystal in contact with them assumes an orientation perpendicular to the surface of the quantum dots. Upon irradiation from a UV light source, the fluorescent spectrum shows a peak at a particular wavelength. When these dots are exposed to the analyte, the liquid crystal undergoes orientational transition, which causes a shift in the peak position.

The present invention is not limited to the use of any particular type of quantum dot. In some preferred embodiments, cadmium selenide quantum dots with thin zinc sulphide and polymer coatings are functionalized with a carboxylic acid terminated organic layer (for example 11-mercaptoundecanoic acid) and then treated to display a recognition moiety as described above (e.g., lead, indium, or gallium perchlorate salts). In some preferred embodiments, the quantum dots are dispersed in a liquid crystal (e.g., 5CB). The functionalized quantum dots align liquid crystals perpendicular to the surface. In further preferred embodiments, a liquid crystal assay device is fabricated by forming a cavity (preferably 5 to 100 microns, most preferably about 25 microns) between two untreated UV transparent quartz substrates. The cavity between the substrates is filled with the mixture of functionalized quantum dots and liquid crystal. In still further preferred embodiments, the optical cell is exposed to an analyte (such as Malathion, or Diazinon) and then probed with a UV light source, such as a laser. When the analyte binds to the receptors on the quantum dots, the liquid crystal aligns parallel to the surface of the quantum dots thereby affecting the color of light emitted by them.

D. Diffraction Based Detection

In still other embodiments, the present invention provides devices and method for standoff detection of analytes that utilize diffraction based probing. The present invention is not limited to any particular mechanism of action. Indeed, an understanding of the mechanism of action is not necessary to understand the present invention. Nevertheless, when a parallel beam of electromagnetic radiation passes through a medium with a periodic refractive index, the beam is diffracted. The diffracted spectrum consists of a central peak and a number of higher order peaks on either side of it. According, in some embodiments, the present invention provides a liquid crystal assay device having at least one surface comprising periodic lines of gold film displaying a recognition moiety and filled with a liquid crystal. In the absence of any analyte, the area not displaying the recognition moiety aligns liquid crystal in homeotropic configuration and the area that is not displaying a recognition moiety aligns randomly in the plane parallel to the substrate. This introduces a periodic structure in the refractive index. When the optical cell is exposed to the analyte, the liquid crystal undergoes orientational transition only in the area displaying the receptors. The liquid crystal assumes random planar orientation in the area displaying the recognition moiety, thereby erasing the periodic refractive index profile. Thus, there is no diffraction effect.

Accordingly, in some preferred embodiments, liquid crystal assay devices are fabricated by depositing a thin film of metal (e.g., about 75 nm of gold) onto a substrate (e.g., glass) using an electron beam evaporator. In further preferred embodiments, narrow gold lines (e.g., about 1 micron in width with about 1 micron spacing) are etched on the surface using conventional photolithography. The substrate is then functionalized with an organic layer as described above. The organic layer only assembles where gold is present. The substrate is then treated to display a recognition moiety as described above. A device having a chamber therein is formed by bonding the treated substrate to another substrate (e.g., glass treated with (Tridecafluoro-1,1,2,2-tetrahydrooctyl)triethoxysilane (OTS)). Preferably, the chamber is about 25 nm thick. The chamber is filled with LC such as 5CB. The liquid crystal aligns perpendicular to the surface only in the region where the receptor chemistry is present. The area that does not have the receptor chemistry aligns randomly. When illuminated with a light source (e.g., a laser source emitting monochromatic light at 630 nm) the incident light is diffracted showing multiple peaks due to the periodic change in the refractive index of the film of the liquid crystal. Since the binding of the analyte to the recognition moiety causes the liquid crystal to assume planar orientation, the periodic variation in the refractive index profile is erased and hence the diffracted spectrum shows a single peak corresponding to the principle maxima. Absence of the higher order diffraction pattern indicates the presence of the analyte.

IX. Kits

In some embodiments, the present invention provides kits for the detection of organophosphates. In preferred embodiments, the kits comprise one or more substrates as described in detail above. In further embodiments, the kits comprise a second substrate and materials for assembling a liquid crystal cells. In some embodiments, the kits comprise a vial containing mesogens. In still other embodiments, the kits comprise at least one vial containing a control organophosphate. In still other embodiments, the kit comprises instructions for using the reagents contained in the kit for the detection of at least one type of organophosphate. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

Experimental

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); U (units), mU (milliunits); min. (minutes); sec. (seconds).

### EXAMPLE 1

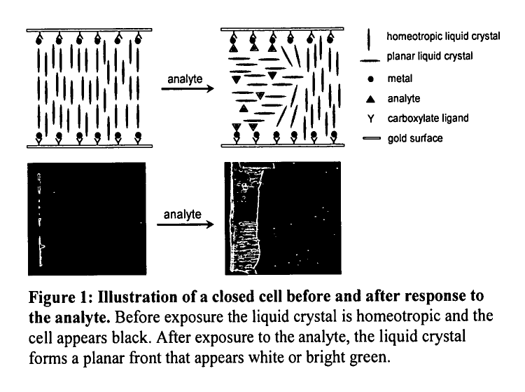

This example describes the identification of metal ion receptors that can be used for detection of OP compounds. This screen was performed by using a “closed” optical cell comprised of two surfaces treated with the metal ions, as shown in FIG. 1. The two surfaces decorated with the metal ions were prepared by (I) depositing ultrathin (optically transparent) gold films onto glass microscope slides by electron beam deposition, (II) forming self-assembled monolayers of mercaptoundecanoic acid (MUA) on the surfaces of the gold films, and (III) immersing the treated gold surface into ethanolic solutions of metal salts to form the metal carboxylates on the surfaces of the films. Uniformly gold coated AlSi glass slides (75 Angstrom gold with 15 Angstrom of titanium) are immersed in to a 1 mM ethanolic solution of MUA and rinsed with Ethanol for 20 seconds and dried with Nitrogen gas. These substrates are cut in to 2.5×2.5 cm squares and spin coated with 1 mM metal perchlorate (35 micro liter) at 3000 rpm for 20 seconds. These square pieces are left to sit out on bench top for 45 minutes for ethanol evaporation. The closed optical cells were formed by pairing two such surfaces, separated by a thin (25 μm) Mylar film, and then filling the cavity between the surfaces with LC. As shown in FIG. 1, the interactions of the LC with the metal ion-decorated surfaces define an orientation of the LC within the cavity of the optical cell. The diffusion of an OP compound from a gaseous phase into the LC leads to a change in the surface-directed orientation of the LC due to the binding of the OP compound to the metal ions on the surface of the cell. The change in orientation of the LC gives rise to a visibly distinct band that accompanies the time-dependent penetration of the OP into the LC. The appearance of the band is caused by the change in optical properties of the LC accompanying the change in orientation. In the absence of OPs, the LC assumes a homeotropic alignment that preserves the plane of polarization of light passing through it. In this situation, little light passes through polarizing filters arranged in a cross polar configuration. When OPs bind to the metal ions, the LC mesogens are displaced and the LC becomes disordered. The greater the degree of disorder present in the LC, the greater the amount of light that passes through polarizing filters (see FIG. 1). The extent to which each metal could report the presence of DMMP was characterized by measuring the distance from the edge of the optical cell (depth of penetration) over which the orientation of the LC was changed upon exposure to DMMP (FIG. 1).

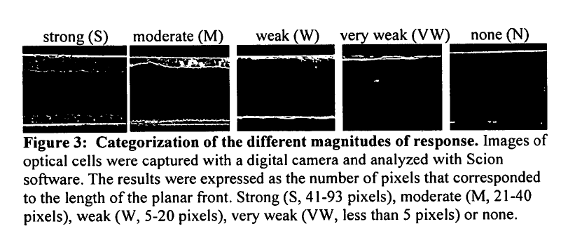

Using the sensing surfaces, decorated with differing metalloreceptors and integrated into optical cells as described above, 17 different metal ions were surveyed for their ability to report the presence of DMMP and other potentially interfering compounds. The results are summarized in Table 1. The results are categorized as illustrated in FIG. 3.

TABLE 1
Qualitative response of optical cells to various compounds at ˜300 ppm. The
cells were fabricated using chemically-treated (1 mM MUA/1 mM metal perchlorate)
gold-coated glass slides. The cells were loaded with E7 liquid crystal.

| | acetic acid | acetone | NH₃OH | DMMP | DMSO | ethanol | ethyl acetate | pentanethiol | pyridine |
|---|---|---|---|---|---|---|---|---|---|
| Al³⁺ | VW | N | M | S | S | N | N | N | W |
| Ba³⁺ | VV | N | W | S | S | N | N | N | N |
| Cd²⁺ | VW | N | VW | S | S | N | N | W | M |
| Ce³⁺ | VW | N | W | S | S | N | N | N | VW |
| Co²⁺ | N | N | N | S | S | N | N | N | VW |
| Cr³⁺ | N | N | M | S | S | N | N | N | VW |
| Cu²⁺ | W | N | N | S | S | N | N | S | M |
| Eu³⁺ | N | N | W | S | S | N | N | N | VW |
| Fe²⁺ | W | N | M | S | S | N | N | N | W |
| Fe³⁺ | W | N | W | S | S | N | N | S | N |
| Ga³⁺ | W | N | S | S | S | N | N | N | S |
| In³⁺ | N | N | S | S | S | N | N | W | S |
| Mn²⁺ | N | N | N | S | S | N | N | N | N |
| Ni²⁺ | N | N | N | S | S | N | N | N | N |
| Pb²⁺ | VW | N | M | S | S | N | N | N | N |
| Pr³⁺ | VW | N | S | S | S | N | N | VW | N |
| Zn²⁺ | S | N | W | S | S | N | N | S | S |

S = strong,
M = moderate,
W = weak,
VW = very weak,
N = none.

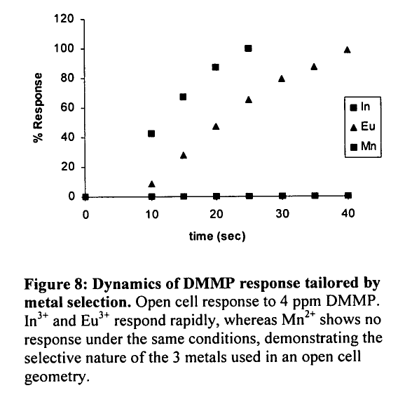

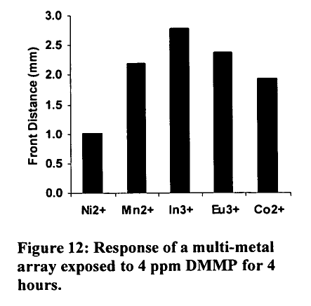

Inspection of Table 1 reveals that all metals generated a strong response to DMMP. In contrast, the other compounds studied showed various patterns of response to the different metal ions. The one exception is DMSO (which is very similar in structure to DMMP), which also showed a strong response across the spectrum of metals studied. Screening of these compounds was done at relatively high concentration (300 ppm). The degree of response of metals to DMMP and DMSO (although all categorized as strong) varied widely among metals. These differences are more pronounced at lower concentrations of each chemical and can be enhanced by altering the methods of optical cell fabrication (please see FIGS. 8 and 12).

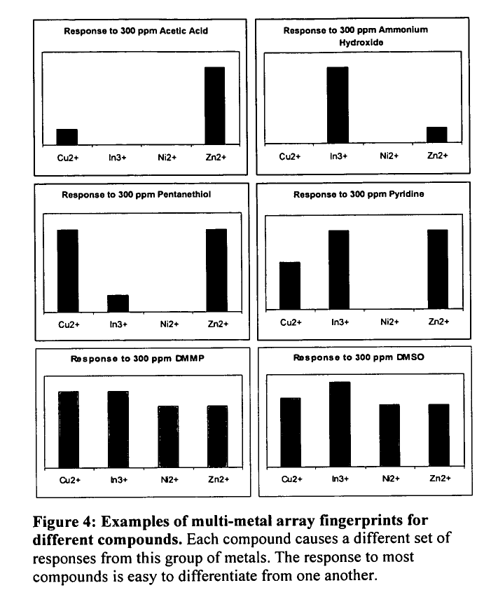

Strategies to discriminate between any interfering compounds are described in more detail below. The patterns of response to the different compounds is illustrated in FIG. 4 by showing in graphical form the patterns generated by 6 compounds using 4 metal ion receptors. These results illustrate the use of combinations of metal ions and the response of the LC on the metal ions to generate signatures that are unique to a given compound.

Because the presence of water is ubiquitous in personal and environmental monitoring, particular effort was devoted to identification of the potential interfering effects of water. Inspection of Table 2 shows that when using the LC E7, 6 of the metals showed no response to water when using 75% humidity and 20 hours of exposure. Only one metal, Zn²⁺, showed a strong response. In addition, it was demonstrated that by selection of the LC, it is possible to make the all metals tolerant to water. As shown in Table 2, when using the LC 8CB, none of the metals responded to the presence of 75% humidity over a 20 hour period.

TABLE 2
Effect of liquid crystal on response to humidity of cells with selected
metals. Optical cells functionalized with different metals were exposed
to 75% humidity for 20 hours. When E7 was used, the sensitivity to high
humidity was dependent on the metal selection. Cells with 8CB showed no
sensitivity to humidity regardless of the metal used.

| | none | very weak | weak | strong |
|---|---|---|---|---|
| E7 | Co²⁺, Cr³⁺, Eu³⁺, | Al³⁺, Cd²⁺, Ce³⁺, | Ba²⁺, Cu²⁺, Fe²⁺, | Zn²⁺ |
| | In³⁺, Mn²⁺, Ni²⁺ | Pb²⁺, Pr²⁺ | Fe³⁺, Ga³⁺ | |
| 8CB | all 18 metals | | | |
| | tested | | | |

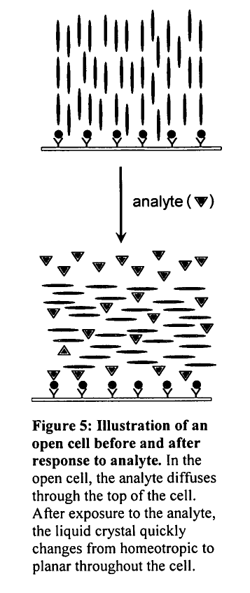

The potential interfering effects of water was further investigated by preparing “open” optical cells. In this case, a thin (˜10 μm) LC film was placed onto an open faced sensing surface decorated with a metalloreceptor. The diffusion path for the DMMP is substantially shortened when using open optical cells, and thus, the temporal response to DMMP is accelerated. A schematic illustration of this experimental geometry is shown in FIG. 5.

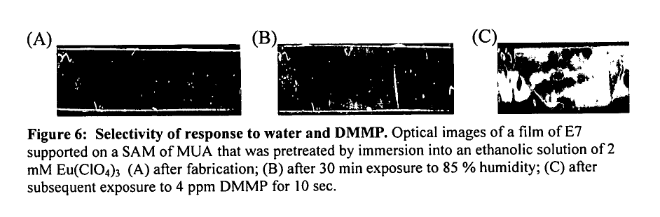

The results of an experiment performed with open optical cells are shown in FIG. 6. Open cells formed using Eu³⁺ that possess high sensitivity to DMMP were exposed to ˜80% humidity for 30 min. The cells showed a very weak response to water with ˜5% of the surface area responding. After exposure to 85% humidity, the cells were re-exposed to 4 ppm DMMP and observed almost the same sensitivity as before exposure to the humidity.

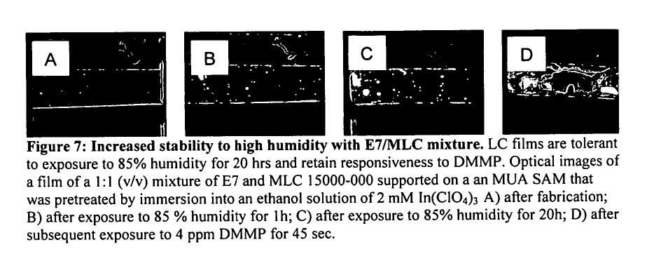

The open cell geometry was also used to demonstrate that the properties of the LC can be tuned to engineer tolerance to interfering compounds such as water. As shown in FIG. 7, a film of a 1:1 mixture of E7 and MLC 15,000-000 was employed, which significantly enhanced stability of the open cell geometry to humidity.

The open cell geometry was also used to investigate the dynamics of the response of the LCs using three metals that all gave strong responses in the closed cell geometry to DMMP (Table 1). The closed cell geometry reports exposure over periods of hours whereas the results obtained using the open cell were obtained over seconds. As shown in FIG. 8, the dynamics of the response of the LC over very short time spans to DMMP is very different for In, Eu and Mn ions. It should be noted that if long term exposures are used with the open cell geometry, Mn (and other metals) will respond in a manner consistent with the data presented in Table 1 using the closed cell geometries.

In summary, metal ions can be selected to tune the selectivity of the LCs to targeted analytes, including OPs. In particular, it is possible to identify sets of metal ions that provide unique responses to compounds. It is also possible to find metal ions that provide strong responses to DMMP but are not influenced by changes in humidity. As described in more detail below, it has been possible to extend these accomplishments to detection of pesticides.

### EXAMPLE 2

This example describes the use of liquid crystal assays for measurement of instantaneous and cumulative exposure to targeted compounds. For these experiments, DMMP was used as a model OP analyte.

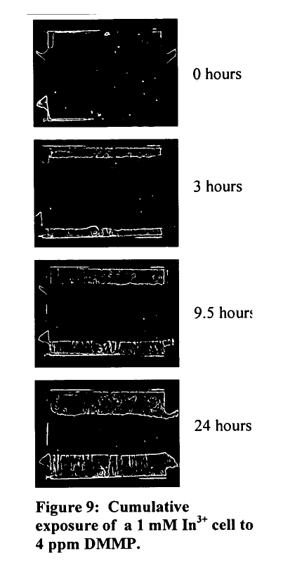

FIG. 9 shows the response of a closed cell to cumulative exposure to DMMP over a 24 hour period. Inspection of FIG. 9 reveals the time-dependent progress of a bright front from the edge of the optical cell towards to the middle. The front is caused by the diffusion of the DMMP into the LC, and the triggering of a change in orientation of the LC, as described in FIG. 1. Measurement of the distance of penetration of the DMMP in the LC provides a quantitative measure of cumulative exposure.

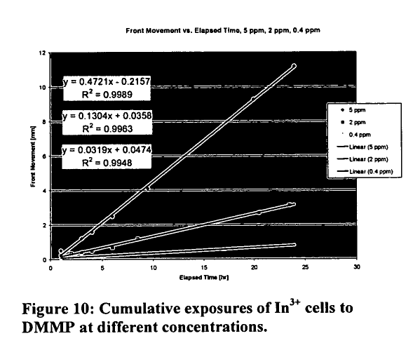

The results shown in FIG. 10 demonstrate that the rate of progress of the front into the LC depends on the concentration of the DMMP. Thus the distance of penetration of the front indicates a convolution of the time and concentration of exposure to DMMP.

An analysis of the results show in FIG. 10 indicates that for all concentrations tested, the data obey a linear fit with a coefficient of correlation >0.99

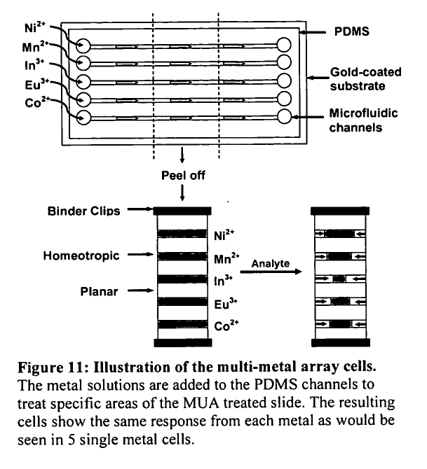

It has also been demonstrated that it is possible to pattern multiple metal ions on surfaces, and thus indicate simultaneously the cumulative response of several metals to DMMP. As described in the results presented in Example 1, the pattern of response of several metal ions can be used to identify particular compounds. FIG. 11 shows a schematic illustration of five metal ions patterned on a surface by using solutions of metal ions delivered to the surface via a microfluidic channel made from PDMS.

The results in FIG. 12 show the different responses of the metal ions to exposure to DMMP for 4 hours. These responses were obtained simultaneously by using the surface patterned with the 5 metal ions.

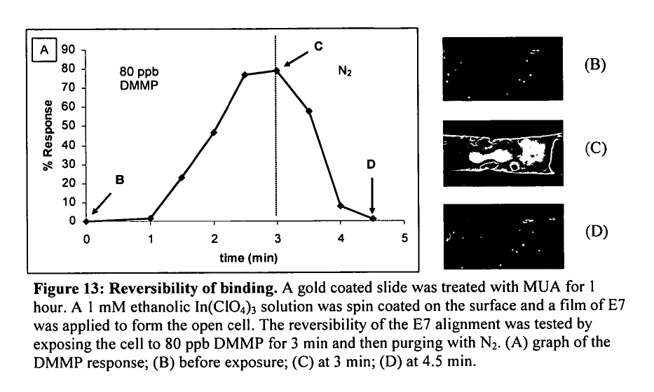

In addition to demonstrating principles for indicating cumulative exposure to an analyte, methods for measuring instantaneous exposure have been demonstrated. These latter experiments were performed using open cells (see FIG. 5). The results shown in FIG. 13 reveal the dynamic response of the LC to exposure to 80 ppb of DMMP. Inspection of this figure shows that the response occurs within a minute of exposure to the analyte, and that the response is completely reversible. It is noted that there exists a 1 minute lag time between the onset of exposure and the initial response of the LC. This lag time is seen in some samples but not all. Some samples (not shown here) have indicated a response within a few seconds of exposure.

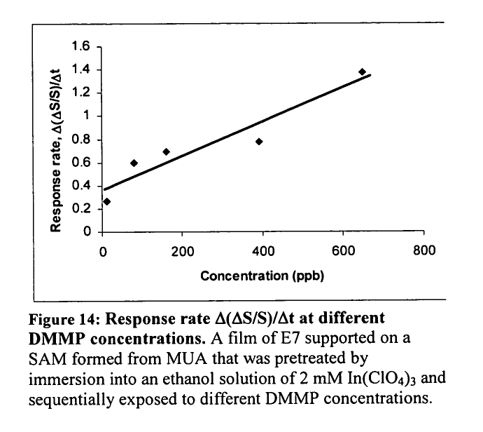

FIG. 14 shows the response rate for the open cells exposed to different DMMP concentrations. This graph indicates that the dynamics of the response can be used to indicate the concentration of DMMP.

These results demonstrate that it is possible to use liquid crystal devices to measure cumulative exposure to DMMP. These principles have been translated to measurement of cumulative exposure to pesticides, as described below in Example 3. In addition, it has been demonstrated that open optical cells can be used to indicate instantaneous exposure to DMMP, with a fully reversible response.

### EXAMPLE 3

This example demonstrates the use of liquid crystal assay devices to measure cumulative exposure to organophosphate-based pesticides. The results in Table 3 summarize a screening of candidate metal ions for their response to Malathion and Diazinon. Because commercial formulations of pesticides contain a number of additives (e.g., surfactants to control wetting), the response of the LC to commercial formulations and purified Diazinon were compared (they were similar). The results in Table 3 indicate the successful identification of metal ions that report both Malathion and Diazinon. Note that the vapor pressures for Malathion and Diazinon are 5.25×10⁻⁵ atm at 30° C. and 9.57×10⁻¹⁰ atm at 20° C., respectively.

TABLE 3
Qualitative response of optical cells to 20 hour pesticide exposure. The cells were fabricated
using chemically-treated (1 mM MUA/1 mM metal perchlorate) gold-coated glass slides. The cells were loaded
with E7 liquid crystal. Five metals, Co²⁺, Cu²⁺, Mn²⁺, Ni²⁺ and Zn²⁺, showed no response to the pesticides.

| | Al³⁺ | Ba²⁺ | Cd²⁺ | Ce³⁺ | Cr³⁺ | Eu³⁺ | Fe²⁺ | Fe³⁺ | Ga³⁺ | In³⁺ | Pb²⁺ | Pr³⁺ |
|---|---|---|---|---|---|---|---|---|---|---|---|---|
| “Store Bought” Malathion | N | N | N | N | N | N | VW | N | N | N | VW | N |
| Malathion solution | N | N | N | N | N | N | VW | N | N | VW | VW | N |
| “Store Bought” Diazinon | M | VW | VW | M | M | M | W | M | S | S | M | W |

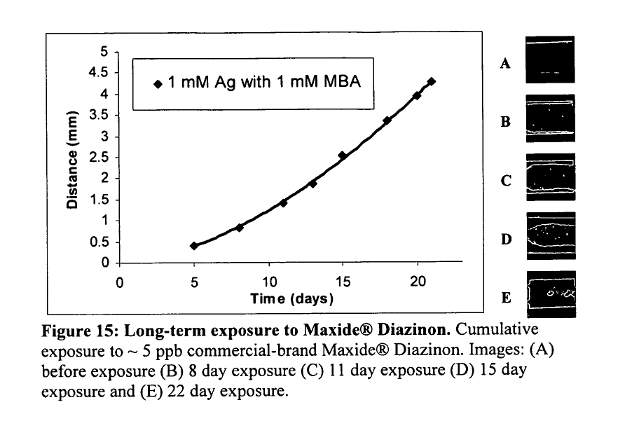

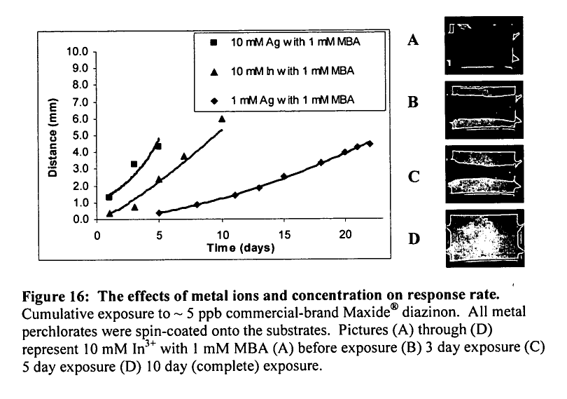

The results in FIG. 15 demonstrate the successful measurement of cumulative exposure to Diazinon over 22 days. Inspection of FIG. 15 reveals a well-defined response by using Ag⁺ ions as the metal receptors. Furthermore, it was possible to tune the time over which cumulative exposure was indicated by using different concentrations of the metal ions when preparing the surfaces. These results are presented in FIG. 16. The results in FIG. 16 indicate that it is possible to tune the maximal response of the LC to Diazinon from 4 days to 22 days by varying the Ag⁺ concentration in the ethanolic solution from 1 mM to 10 mM.

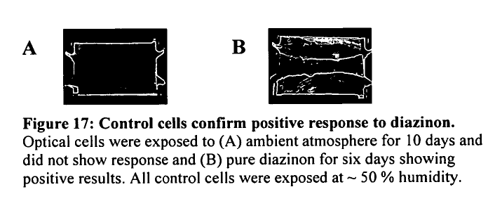

Control experiments were performed to demonstrate that the time-dependent responses reported above were indicators of exposure to Diazinon, and not laboratory contaminants. The results in FIG. 17 demonstrate one such control experiment.

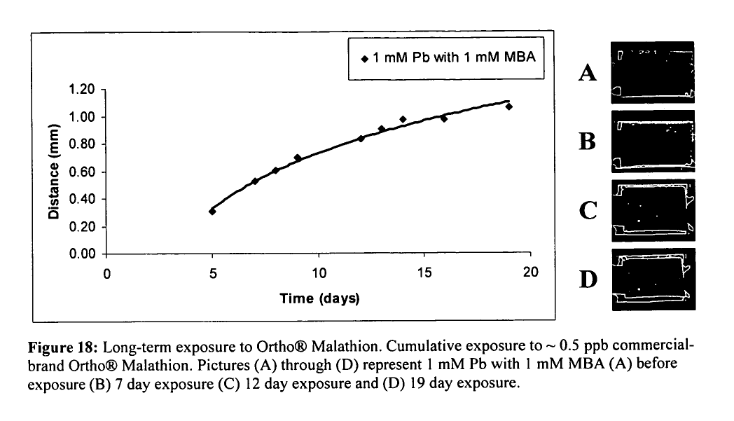

FIG. 18 demonstrates the cumulative measurement of a commercial preparation of Malathion at a low (0.5 ppb) concentration.

In summary, these results demonstrate the use of liquid crystal assay devices to assay cumulative exposure to OP pesticides. Furthermore, parameters have been identified that permit the response of the monitor to be tuned to the length of the desired sampling period, from days to weeks.

### EXAMPLE 4

This example describes the assessment of the potential for environmental compounds to interfere with the ability of the liquid crystal assay devices to detect exposure to organophosphate pesticides. The results described above led to the successful identification of 5 metal ions—namely, In³⁺, Eu³⁺, Mn²⁺, Ni²⁺, and Co²⁺—that were selected on the basis of sensitivity to DMMP (model OP) and tolerance to non-targeted compounds (acetic acid, ammonium hydroxide, dimethylsulfoxide, ethanol, ethylacetate, pentanethiol and pyridine) for use as metalloreceptors in the liquid crystal assay devices. Metal ion receptors were identified that were capable of reporting cumulative exposure to Diazinon (e.g., Ag⁺) and Malathion (Pb²⁺). Metalloreceptors for three OP-based pesticides (Diazinon, Malathion and Parathion) will be identified by additional screening of metalloreceptors capable of reporting cumulative exposure to the pesticides and as well as possessing tolerance to non-targeted compounds. In particular, screening will be conducted for interfering compounds and mixtures of compounds that are likely to be encountered in residential and agricultural environments in which the monitors will ultimately be used.

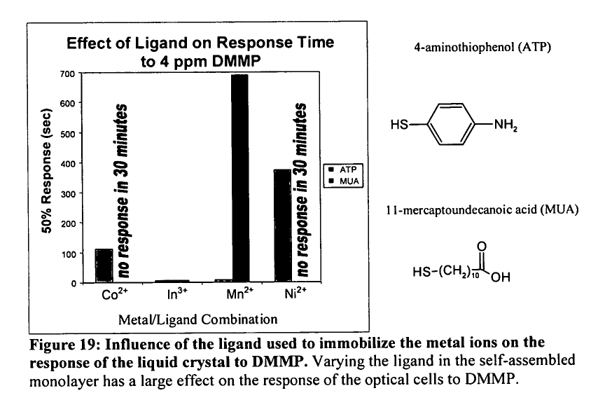

Metal ions receptors will be identified that can report the presence of one or more of three OP-pesticides (Diazinon, Malathion and Parathion) without interference from compounds likely to be encountered in residential or agricultural environments. Some of these compounds are complex mixtures and include: exhaust from combustion engines, kitchen odors, wood smoke, perfume, gasoline, diesel fuel, ammonia, fertilizer, baby lotion, hair spray, nail polisher (acetone), insecticides, cigarette smoke, NOₓ, CO, floor cleaners, furniture polish and household deodorizers.

Whereas the examples described above utilized mercaptoundecanoic acid (MUA) as the ligand with which to immobilize the metal ions on surfaces, studies performed indicate that the choice of the ligand can be exploited to tune the sensitivity and selectivity of the response of the LC to the OPs (FIG. 19). Thus, the influence of the choice of metal ion receptors for OP-pesticides will be evaluated in combination with choice of three ligands (4-aminothiophenol (ATP), mercaptobenzoic acid (MBA) and MUA. Note that all these ligands possess a mercaptan functionality, and thus can be assembled on the surface of ultra-thin gold films by virtue of the strong binding of mercaptans to gold (leading to so-called self-assembled monolayers). The metal ions will be immobilized via their interactions with amino (ATP) or carboxylic acid groups (MUA and MBA) of these ligands.

The experiments described below, will make use of closed LC optical cells of the type shown in FIG. 1. These cells will be fabricated using methods identical to those described above. In short, two surfaces decorated with the metal ions will be prepared by (I) depositing ultrathin (optically transparent) gold films onto glass microscope slides by electron beam deposition, (II) forming self-assembled monolayers of a ligand for metal ions (e.g., MUA) on the surfaces of the gold films, and (III) immersing the treated gold surfaces into ethanolic solutions of metal salts to form the metal receptors on the surfaces of the films. The optical cells will be formed by pairing two such surfaces, spacing them apart using a 25 μm thick film of Mylar, and then filling the cavity between the surfaces with LC. The diffusion of the OP pesticide into the LC will lead to a change in the surface-directed orientation of the LC due to the binding of the OP pesticide to the metal ions on the surface of the cell (FIG. 1). The change in orientation of the LC will give rise to a visibly distinct band in the LC that accompanies the time-dependent penetration of the OP pesticide into the LC. The distance from the edge of the optical cell (depth of penetration) over which the orientation of the LC is changed upon exposure to OP pesticide will be measured (see below for additional details regarding the methods of analysis).

The following sequence of experiments will be performed utilized these assay devices:

a. Self-assembled monolayers from ATP, BMA and MUA will be formed on the surfaces of ultra-thin gold films deposited using the methods described above.

b. Metal ions will be patterned on these surfaces using the procedures described in FIG. 12. Initially, Ni²⁺, Mn²⁺, In³⁺, Eu³⁺, Co²⁺, Ag¹⁺ and Pb²⁺ will be patterned on the surfaces of the treated gold films. Concentrations of metal ions between 1 mM and 100 mM will be used when binding the metal ions to the ligands. Choice of the metal ion receptors will also be guided by concepts of hard/soft acids and bases. In these experiments, the metal ions are the Lewis acids (electron acceptors) and the targeted compounds are Lewis bases (electron donors). As first described by Pearson in the 1960's, hard acids (small, compact, highly charged electron pair acceptors) typically bind strongly to hard bases (small, highly electronegative electron-pair donors), and soft acids bind strongly to soft bases (soft acids and bases are large, diffuse and polarizable species). Because the oxygens of OP compounds and the nitrogens of organonitrogen compounds are hard bases, an initial screen will be conducted for strong binding between these compounds and hard metals (such as those listed above). Additional factors that may lead to discrimination between compounds include molecular shape (shape accommodation) and degree of polarity.

c. Using the methods of exposure described in Example 7 below, screens will be conducted for metals that provide indications of cumulative exposure to Parathion, Malathion and Diazinon for periods between 1 day and 3 weeks. These initial screens will be performed at saturated vapor concentrations of the pesticides. The results above indicate that Ag¹⁺ in combination with MBA provides a useful indicator of cumulative exposure to Diazinon (FIG. 16). In addition, Pb²⁺ in combination with MBA provides a useful indicator of cumulative exposure to Malathion (FIG. 18). Technical grade pesticides will be used in these initial studies with commercial residential products subsequently evaluated.

d. Metal/ligand combinations that are demonstrated in “c” to indicate cumulative exposure to at least one of the three pesticides will be screened for interference to exhaust from internal combustion engines, kitchen odors, wood smoke, perfume, gasoline, diesel fuel, fertilizer, ammonia, baby lotion, hair spray, nail polisher (acetone), insecticides, cigarette smoke, NOₓ, CO, floor cleaners, furniture polish and household deodorizers.

e. Metal/ligand combinations in “d” above that are found not to respond to potentially interfering compounds in isolation will be subsequently screened for the influence of the potentially interfering compounds on the response of the metal ion/ligand to the three OP pesticides. This experiment will be performed by comparing the response of the LCs to the pesticides in the presence and absence of the potentially interfering compounds.

### EXAMPLE 5

This example describes testing for the potential influence of degradation products of OP pesticides on the response of the liquid crystal assay devices. When OP compounds are exposed to the atmosphere, they are frequently oxidized or hydrolyzed (Bavcon et al, 2002). Malathion is oxidized to malaoxon while the major degradation of Diazinon is by hydrolysis to form 2-isopropyl-6-methyl-4-pyrimidinol (IMP). Tests will be conducted to determine the response of the metal ion/ligand combinations identified above to the presence of these two degradation products in stored samples of pesticide and to determine their influence on the detection of Malathion and Diazinon.

A mixture of known concentrations of Malathion and Diazinon in water will be prepared and stored for 1, 3, 5, 7 and 14 days prior to measurement of the response of the liquid crystal assay device. The vapor will be generated using methods described below. The data obtained from each exposure will be compared to that obtained to OPs that are not stored and vapors prepared using malaoxon and IMP. The percent of degradation product present in the aqueous solutions at each time point will be determined by separation of the pesticides and their two metabolites on an SPB-1 column as described by Bavcon et. al.

### EXAMPLE 6

This Example describes additional strategies to diminish and eliminate the impact of interfering environmental chemicals on detection of OPs by the liquid crystal assay devices. The result above demonstrated that the sensitivity and selectivity of the liquid crystal assay devices can be tuned substantially by the choice of the metal ion and the magnitude of response is modulated by the concentration of the metal ion immobilized on the nanostructured substrate. As described above, the ligand hosting the metal ion on the surface to influence the sensitivity and selectivity has also been identified. In preliminary experiments, the influence of the identity of the LC on the tolerance of the system to the potentially interfering compounds has been explored. The choice of the LC provides another useful degree of freedom through which to minimize the effects of potentially interfering compounds. For example, a mixture of the LCs E7 and MLC 15,000-000 significantly enhanced stability of the open cell to humidity without changing response to DMMP. Furthermore, a film of 8CB gives no response to humidity. Optimization of the LC depends on the nature of the potentially interfering compounds identified above, and will be guided by Brønstead acid-base theory. Potentially interfering compounds will be classified as protic (e.g., amines, alcohols, carboxylic acids), dipolar aprotic (e.g., DMSO, CH₃CN, CH₃NO₂, phosphate, CH₂Cl₂) or apolar aprotic (e.g., benzene, alkanes). For example, if a film of LC is designed to be hydrophobic, the penetration of protic compounds, where hydrogen bonding is important, into the film will significantly diminish, as observed the experiments using E7 and MLC 15,000-000.

A second approach that can be evaluated to minimize the effects of potentially interfering compounds will exploit polymeric membranes across which the OP must diffuse. It is noted that that polymeric membranes have been widely used in sensors of gas phases to enhance selectivity (Digest of the 14^{th} Chemical Sensor Symposium, 1992). For instance a polydimethylsiloxane (PDMS) membrane is hydrophobic and does not allow the passage of water but will permit the passage of less polar compounds (e.g., OPs). It is noted that a wide range of membrane materials for separation and purification of gas phases do exist. Finally, it is noted that insertion of a thin strip of a membrane material between the two surfaces of the cell shown in FIG. 1 is technically straightforward, and would provide additional protection of the LC from dust and other foreign material.

### EXAMPLE 7

This example describes preferred methods for aerosol generation and measurement for the series of experiments performed at the University of Minnesota laboratories. All laboratory experiments will be conducted in a lab hood using a fabricated sampling chamber constructed of an inert material. For initial experiments measuring gas-phase pesticides, concentration levels will be determined based on physical properties and practical considerations, such as lower limit of measurable concentrations for the liquid crystal assay devices, safety of laboratory personnel and feasibility. Malathion, for example, has a saturated vapor pressure of 5.3 mPa at 30° C. Therefore, the maximum concentration of Malathion in the vapor phase will be about 50 ppb. Any additional Malathion will exist as droplets or will condense onto other particles or surfaces.

Gas-phase concentrations will be generated by bubbling air through technical grade Malathion/Diazinon to saturate the air, which will then be filtered to remove aerosols and mixed with a dilution gas stream to achieve desired gas-phase concentrations. Relative humidity (RH) levels (15, 50, 85%) will be achieved by passing dilution air through a bubbler or sorbants, as appropriate to achieve desired conditions, and data on RH and temperature collected using a continuous monitor.

Aerosol-phase experiments will use these same active ingredients. Both Diazinon and Malathion have several formulations for homeowners use outdoors, however, but most are applied using hand operated sprayers, such as aerosol cans, hose-end, or compressed air sprayers. Pesticide aerosols will be generated using the sampling chamber and dilution stream set up described above, but aerosols will be generated upstream and introduced directly into the chamber. The laboratory has several compressed air nebulizers that produce aerosols of uniform small particle sizes by removing larger spray droplets by impaction within the device. Final choice of nebulizer will be made to match the particle size produced by representative hand-operated sprayers, depending on the specific pesticide formulations tested. This will be determined early in the series of experiments, with the aim of choosing a well characterized aerosol generator applicable to a wide variety of conditions, formulations, and active ingredients NIOSH method 5600, which uses sorbant tubes (quartz fiber filters, backed with a XAD/PUF/XAD sandwich) will be used to collect duplicate reference measurements for both gas-phase and aerosol measurements during each experiment. We will conduct additional experiments to determine if use of this method results in unacceptable sample loss by comparing these results to those obtained using an electrostatic precipitator, a device for collecting airborne particles on charged plates.

OP compounds will be measured using the Agilent Model 6850 Gas Chromatograph (GC) with a flame ionization detector in the Division of Environmental and Occupational Health Laboratories at University of Minnesota. We will analyze all pesticide samples that we collect via sorbants using NIOSH method 5600 (NIOSH 1994). Test compound will be extracted using a 90% toluene/10% acetone solution, and temperature and carrier gas flows will follow standard operating parameters in the laboratory.

### EXAMPLE 8

This Example describes standardization of the format for the liquid crystal assay device. The method of analysis used above comprised the following procedure: (I) fabricate an optical cell ˜1″×0.5″ with Mylar spacer on the ends; (II) place optical cell between cross-polars lit by light box. Acquire image with camera at defined distance, aperture and shutter speed; (III) reduce size to 800 by 600 pixels and convert to bmp file format; (IV) using Scion Image (NIH freeware), manually measure the area of the wavefront and its length to calculate the distance of penetration of the OP into the LC in units of “pixels”; and then (V) convert units of “pixels” to “mm” using an image of a 1 mm scale-bar inserted into the image when the image of the LC cell was acquired (Step II).

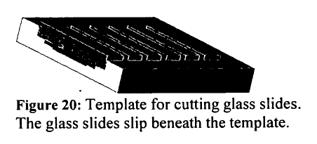

Devices will be produced by standardizing cell dimensions to 0.5″×1″ and assemble a simple template that permits precision cutting of glass slides to this standard size (tolerance of 50 micrometers). A template similar to FIG. 20 will be fabricated to ensure cuts perpendicular to the edge of the glass slide at 0.5″ intervals. The edges of the top and bottom surfaces of the LC cell will be aligned within a tolerance of 5 micrometers to create a reproducible geometry at the edge of the LC cell at which the OP enters the LC.

Procedures used to acquire the images of the cells will be standardized. This procedure will utilize a monochromatic light source (660 nm LEDs) with uniform light distribution (Edmond Scientific). This wavelength lies in the optimal range for reporting changes in LC alignment. The camera settings will be standardized to ensure exact image replicas each time an image of the cell is acquired. The focal plane, shutter speed and aperture of the fixed focal length Cosmicar® lens will be set to produce the same image acquisition conditions each time. By creating the same exact imaging conditions each time, it will make possible automation of the analysis of the response of the LC to the OP pesticides.

Next, the image of the cell will be limited to a defined portion of the cell. This procedure will utilize a black mask, placed over the optical cell, that has a defined aperture and is centered over the cell to ensure uniform imaging of the optical cell and to decrease stray light contamination of the image.

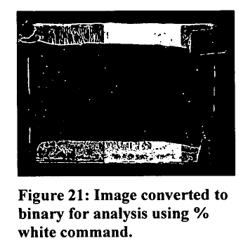

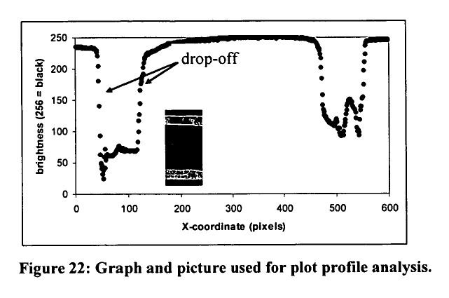

The analysis of the size of the wavefront (response) will also be automated. Using the conditions defined above, two different methods will be tested for the best correlation to analyte concentration. Scion Image (NIH Freeware) or a similar program will be used to convert the images to binary code and then measure the % white area (FIG. 21). Since the cells will be the same size, the % white area can be easily converted to the front distance using the same formula each time. Scion Image will be used to create a profile of the light intensity across the cell and measure the distance between the drop-off points at the edges of each front (FIG. 22). In this process, the pixel on the drop-off gives the most accurate representation of the location of the front will be determined.

### EXAMPLE 9

This example describes stand off detection using a liquid crystal assay device comprising at least two reflective surfaces. Small (˜100 micron in size) Fabry-Perot filters are dispersed in a closed chamber. The size of these filters is small enough so that they tumble in the chamber. A broadband source of light that emits electromagnetic radiation in the near infrared to visible region is directed toward the distribution of these filters. The reflected light is collected by a collimating system and analyzed by a spectrophotometer. In the absence of the target analyte in the chamber, the reflected intensity shows peak at a wavelength that corresponds to the ordinary refractive index of the LC used. The target analyte is then released into the chamber. As soon as the target analyte binds to the receptors on the surface of each of the mirrors, the liquid crystal undergoes orientational transition thereby inducing a shift in the peak position of the reflected spectrum.

### EXAMPLE 10

This example describes stand off detection using a liquid crystal assay device comprising porous silicon. The porous silicon liquid crystal assay device (fabricated as described above) is broken in to small pieces (˜100 micron) by ultrasonication (e.g., to form smart dust). These pieces are dispersed in a closed chamber in which the target analyte can be released in a controlled fashion. A broadband light source in the near IR to visible region is directed to the distribution of these particles and the reflected light is collected by a collimating optics and is analyzed by spectrometer at distance. A peak corresponding to the radial distribution of the liquid crystal appears in the reflected spectrum. Once the target analyte is released into the chamber, it binds to the receptors attached to the wall of the silicon pores and induces an orientational transition in liquid crystal. This causes a shift in the peak position of the reflected light.

### EXAMPLE 11

This example describes stand off detection using a liquid crystal assay device comprising porous silicon and using a UV light source for irradiation. As above, porous silicon liquid crystal assay device is broken in to small pieces (˜100 micron) by ultrasonication. These pieces are distributed in a closed chamber in which the target analyte can be released in a controlled fashion. These particles are exposed to UV light from a laser through a small quartz window that is nearly UV transparent. The illuminated light is collected using a collimated optics and analyzed using a UV-visible photospectrometer located at distance. A peak corresponding to the radial distribution of the liquid crystal appears in the photolumiscence spectrum. Once the target analyte is released into the box, it binds to the receptors attached to the wall of the silicon pores and induces an orientational transition in LC. This causes a shift in the peak position of the reflected light.

### EXAMPLE 12

This example describes stand off detection using a liquid crystal assay device comprising fluorescent moieties. For the field experiments, small (˜100 micron in size) liquid crystal assay devices comprising fluorescent moieties are dispersed in a closed chamber. A laser that emits light at the excitation wavelength of the fluorescence molecules illuminates the distribution of these particles. The fluorescence light emitted by these fluorescence molecules is collected by a collimating optics and analyzed by a spectrophotometer. In the absence of the target analyte in the chamber, the emitted spectrum exhibits peak intensity at a wavelength that corresponds to the ordinary refractive index of the liquid crystal used. The target analyte is then released into the chamber. As soon as the target analyte binds to the receptors on the surface, the liquid crystal undergoes orientational transition thereby inducing a shift in the peak position of the fluorescent spectrum.

### EXAMPLE 13

This example describes stand off detection using a liquid crystal assay device comprising quantum dots. Small (˜100 micron in size) devices are dispersed in a closed chamber. A laser that emits light at the excitation wavelength of the quantum dots illuminates the distribution of these particles. The fluorescence light emitted by the quantum dots is collected by a collimating optics and analyzed by a spectrophotometer. In the absence of the target analyte in the chamber, the emitted spectrum will show peak intensity at the detector that corresponds to the ordinary refractive index of liquid crystal used. The target analyte is then released into the chamber. As soon as the target analyte binds to the receptors on the surface of quantum dots, the liquid crystal undergoes orientational transition thereby inducing a shift in the peak position of the reflected spectrum.

In another embodiment of the same principle, the cadmium selenide quantum dots are functionalized with the carboxylic acid and the receptor chemistry. These particles are then coated with a polymeric liquid crystal. The polymeric liquid crystal aligns perpendicular to the surface of the quantum dots in the absence of any analytes. Upon exposure to the ultraviolet light these quantum dots emit light with the characteristic wavelength that can be monitored from a remote location. When these quantum dots are exposed to analyte, the liquid crystal changes orientations to planar configuration and the wavelength of the light emitted by these quantum dots shifts.

### EXAMPLE 14

This example describes stand off detection using a liquid crystal assay device comprising periodic lines. Small (˜100 micron in size) liquid crystal assay devices comprising periodic lines are dispersed in a closed chamber. A parallel monochromatic beam of light at 630 nm is directed to the ensemble. Two photodetectors are placed at the position of the primary peak and the first order diffracted beam with respect to the incident beam. The relative intensities of the first order diffracted beam and the primary maxima is compared. In the absence of the target analyte in the chamber, the ratio will have a non-zero value less than unity. The target analyte is then released into the chamber. As soon as the target analyte binds to the receptors on the gold lines, the liquid crystal changes orientation to the planar configuration erasing the periodicity in the refractive index. The ratio of the first order diffraction intensity to the principle maxima vanishes to zero indicating the presence of the analyte in the system.

In this particular embodiment, the field experiment can also be performed using different approach. An optical cell is located at a fixed position (such as at the top of a transmission tower in a field) and is periodically interrogated by illuminating with a laser beam. Two detectors are located at fixed locations to detect the diffracted light. The first detector is located at the position of the principle maxima and the second at the position of the first order maxima. The ratio of the intensity of the first order and the principle maxima are compared periodically. As the analyte binds to the receptor on the gold surface the intensity at the second detector goes to zero. This allows passive monitoring of the presence of target analyte in the environment.

What is claimed is:

1. A method of remotely detecting an analyte comprising:
a) providing a plurality of liquid crystal assay devices comprising a first surface displaying a recognition moiety, said first surface in contact with a liquid crystal;
b) exposing said plurality of liquid crystal assay devices to a sample suspected of containing said analyte; and
c) simultaneously irradiating said plurality of liquid crystal assay devices under conditions such that radiation returned from said plurality of liquid crystal assay devices is indicative of a change in orientation of said liquid crystal in said assay devices caused by interaction of said analyte with said recognition moiety.

2. The method of claim 1, wherein said irradiating is performed by exposure to electromagnetic radiation.

3. The method of claim 1, wherein said electromagnetic radiation is selected from the group consisting of visible light, x-ray radiation, UV radiation, infrared radiation, and radiofrequency radiation.

4. The method of claim 1, wherein said radiation returned from said assay device is measured by a method selected from the group consisting of infrared spectroscopy, raman spectroscopy, x-ray spectroscopy, visible light spectroscopy, ultraviolet spectroscopy, spectroscopy of radiofrequency radiation, and combinations thereof.

5. The method of claim 1, wherein said radiation returned from said device exhibits a peak wavelength that is different in the presence of an analyte than in the absence of an analyte.

6. The method of claim 1, wherein said radiation returned from said device exhibits a spectrum that is different in the presence of an analyte than in the absence of an analyte.

7. The method of claim 1, wherein said radiation returned from said device exhibits a change in the intensity of the peak of the spectrum emitted from said device.

8. The method of claim 1, wherein said liquid crystal comprises mesogens selected from the group consisting of E7, MLC, 5CB (4-n-pentyl-4′-cyanobiphenyl), and 8CB (4-cyano-4′octylbiphenyl).

9. The method of claim 1, wherein said liquid crystal assay devices are irradiated by a remote radiation source.

10. The method of claim 9, wherein said remote radiation source is greater than 10 meters from said liquid crystal assay device.

11. A system for remotely detecting an analyte comprising:
a) a plurality of liquid crystal assay devices comprising a first surface displaying a recognition moiety, said first surface in contact with a liquid crystal;
b) a radiation source remote from said plurality of liquid crystal assay devices; and
c) a detector configured to receive a signal from said plurality of assay devices upon radiation of said plurality of assay devices by said radiation source.

12. A method of assaying cumulative exposure to organophosphates comprising:
a) providing a device comprising a liquid crystal, said liquid crystal between a first surface and a second surface, said first surface comprising an organic layer in contact with said first surface, said organic layer having immobilized thereon at least one metal ion, said device having an opening therein; and
b) exposing said device to a sample suspected of containing organophosphates, wherein cumulative exposure to organophosphates is indicated by a change in the orientation of said liquid crystal identified as wavefront advancing from said opening.

13. The method of claim 12, wherein said organic layer has immobilized thereon a plurality of different metal ions selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺.

14. The method of claim 12, wherein said metal ions are arranged in an array.

15. A method of identifying a particular organophosphate comprising:
a) providing a substrate comprising at least two detection regions having at least two different metal ions immobilized thereon; and
b) exposing said device to a sample suspected of containing an organophosphate; and
c) determining the identity of said organophosphate by examining the change of liquid crystal orientation in said detection regions.

16. The method of claim 15, wherein said liquid crystal overlaying said detection regions is disordered in the presence of organophosphates.

17. The method of claim 15, wherein said detection region has immobilized thereon a plurality of different metal ions selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, and Zn²⁺.

18. A device comprising at least a first substrate having a surface, said substrate comprising at least first and second detection regions on said surface, wherein said first and second detection regions comprise an organic layer and a metal ion immobilized on said organic layer and wherein said metal ions on said first and second detection regions are different.

19. A device comprising:
at least a first substrate having a surface, said substrate further comprising at least a first detection region on said surface, wherein said detection region comprises a recognition moiety;
a liquid crystal in contact with said first substrate; and
a housing having an opening therein, said substrate configured in said housing so that said detection region is exposed to the atmosphere through said opening.

20. The device of claim 19, wherein said housing is movable between an exposure position wherein said detection region is exposed to the atmosphere through said opening and a reading position wherein said detection region is substantially closed off to the atmosphere.
